(19) United States
(12) Patent Application Publication
(10) Pub. No.: US 2023/0026199 A1
(43) Pub. Date: Jan. 26, 2023

(54) INORGANIC SUNSCREEN ACTIVE AGENTS IN COSMETIC COMPOSITIONS AND METHODS

(71) Applicant: VIZOR, LLC, Middlesex, NJ (US)

(72) Inventors: Yannick RIGG, Springfield Gardens, NY (US); Richard RIGG, Richmond Hills, NY (US)

(73) Assignee: VIZOR, LLC, Middlesex, NJ (US)

(21) Appl. No.: 17/951,988

(22) Filed: Sep. 23, 2022

Related U.S. Application Data
(60) Provisional application No. 63/300,739, filed on Jan. 19, 2022.

(51) Int. Cl. A61K 8/02; A61K 8/25; A61K 8/27; A61K 8/29; A61K 8/31; A61K 8/365; A61K 8/58; A61Q 17/04

(52) U.S. Cl. CPC A61K 8/025; A61K 8/25; A61K 8/27; A61K 8/29; A61K 8/31; A61K 8/365; A61K 8/585; A61Q 17/04; A61K 2800/412; A61K 2800/622; A61K 2800/623; A61K 2800/651

(57) ABSTRACT

Inorganic sunscreen active agents in cosmetic compositions and methods are provided and feature micronized metal oxide inorganic particles selected from the group consisting of zinc oxide, titanium oxide, cerium oxide and mixtures thereof, the inorganic particles being coated with:
(i) a poly[C8-C20 hydroxycarboxylic acid], the coated particles measured at a 10% loading in dodecane and at 1 minute elapsed time having a Zeta Potential ranging from 2 to 10 mv, particularly from 2 to 5 mv, amounts of the poly[C8-C20 hydroxycarboxylic acid] to the inorganic particles being in a relative weight ratio of 1:100 to 1:10; or alternatively
(ii) an adduct of a hydroxy cinnamate and a silanol, amounts of the adduct to the inorganic particles being in a relative weight ratio of 1:200 to 1:4; or alternatively
(iii) mixtures of (i) and (ii).

### CROSS REFERENCE TO RELATED APPLICATIONS

This application relates to, and claims priority from, U.S. Prov. Ser. No. 63/300,739 filed Jan. 19, 2022, the entire contents of which are fully incorporated herein by reference.

This application also relates to, but does not claim priority from, U.S. Ser. No. 16/638,165 filed Feb. 11, 2020 entitled Inorganic Sunscreen Agents with Higher UV Radiation Protection, and from U.S. Ser. No. 16/638,175 filed Feb. 11, 2020 entitled Hydroxy Cinnamate and Silanol Adduct Coated Inorganic Sunscreen Agents, the entire contents of each of which are incorporated herein fully by reference.

### FIGURE SELECTED FOR PUBLICATION

None

### BACKGROUND OF THE INVENTION

### Field of the Invention

The invention relates to inorganic metal oxide particles useful as sunscreen active agent components for skin and hair care compositions.

### Description of the Related Art

Sunlight can be seriously damaging to human skin and destructive to hair. Ultraviolet segments of sunlight are known to accelerate photo aging of the human dermis. Acute exposure may even cause painful erythema. For these reasons, cosmetic chemists often combine organic sunscreen agents into their formulations. The spectrum of UVA, UVB and Blue Light radiation ordinarily is addressed by cocktails of two or more organic sunscreen agents.

Unfortunately, organic type sunscreen agents suffer from certain problems. Under the bombardment of ultraviolet radiation, the organic sunscreens themselves degrade. Photostability may last for only a few hours. Consumers thinking that they are fully protected with their sunscreen lotion, often expose themselves for a time beyond the photostability limit. A second problem is that prominent organic sunscreen agents under certain conditions are prone to cause skin irritation.

In recent times, microfine zinc oxide and microfine titanium oxide have been shown to deflect ultraviolet radiation of both UVA and UVB type. No longer is photostability and skin irritation a problem. But with any new technology, issues do arise. One problem is formulation space. There is a limit to how much metal oxide particles can be suspended within a cosmetic composition. Ways are needed to raise the sun protective factor (SPF) while keeping constant the level of metal oxide particles.

Background literature includes the following disclosures. U.S. Pat. No. 5,587,148 (Mitchell and Mitchnik) discloses as sunscreen agent a dispersion of micronized particles of zinc oxide with diameter of less than 0.2 micron (200 nm).

U.S. Pat. No. 8,623,386 B2 (Schlossman et al) describes coated metal oxide particles used as pigments in cosmetic compositions. These particles are reported available in primary particle size less than 200 nm and also as pigmentary grade with sizes larger than 200 nm. Coatings are preferably jojoba ester but may also be selected from soya wax, candelilla wax, castor oil, coconut oil, macadamia nut oil, and even many fractions of mineral oil.

U.S. Pat. No. 9,254,398 (Schlossman et al) discloses a series of two-layer coated micronized metal oxide powders with good self-dispersibility. These are intended as ingredients for cosmetic products such as makeup, lipstick, nail enamel, eye shadow and mascara. The first of the two layers is triethoxycaprylylsilane. The second, an outer coating, is polyhydroxystearic acid.

Commercial dispersing agents for sunscreens under the trademark DISPERSUN® are marketed by Innospec Inc. The company in a product brochure (Issue No. 4/2010) promotes use of DISPERSUN® DSP-OL100 and DSP-OL300 (both identified as polyhydroxystearic acid) for dispersing ultrafine titanium dioxide and zinc oxide into sun protection cosmetic products. Higher sun protection factor (SPF) sunscreen was said to be achievable without increasing pigment levels. There is room for more improvement.

### ASPECTS AND SUMMARY OF THE INVENTION

According to one alternative and adaptive aspect of the present invention there is provided an ultraviolet radiation sun protective composition featuring micronized metal oxide inorganic particles selected from the group consisting of zinc oxide, titanium oxide, cerium oxide and mixtures thereof, the inorganic particles being coated with:
(i) a poly[C8-C20 hydroxycarboxylic acid], the coated particles measured at a 10% loading in dodecane and at 1 minute elapsed time having a Zeta Potential ranging from 2 to 10 my, particularly from 2 to 5 my, amounts of the poly[C8-C20 hydroxycarboxylic acid] to the inorganic particles being in a relative weight ratio of 1:100 to 1:10; or
(ii) an adduct of a hydroxy cinnamate and a silanol, amounts of the adduct to the inorganic particles being in a relative weight ratio of 1:200 to 1:4; or
(iii) mixtures of (i) and (ii).

According to other alternative and adaptive aspects of the present invention include or may be adapted to be incorporated with or modified by: Coatings Based on (i) Poly[C8-C20 hydroxycarboxylic acid]; Coatings Based on (ii) an Adduct of a Hydroxy Cinnamate and a Silanol; Metal Oxide Inorganic Particles, Cosmetic Products, Oils/Emollients and Humectants as carriers, Thickeners, Organic UV Filters; other film formers, emulsifiers and surfactants, and others within the scope and spirit of the present invention.

The above and other aspects, features and advantages of the present invention will become apparent from the following description read in conjunction with the accompanying disclosure and formulations.

### BRIEF DESCRIPTION OF THE DRAWINGS

No drawings are provided herein.

### DETAILED DESCRIPTION OF THE PREFERRED EMBODIMENTS

Reference will now be made in detail to adaptive compositions, methods, and embodiments of the invention. It will also be understood that other embodiments may be utilized without departing from the scope of the present invention, and that the detailed description is not to be taken in a limiting sense, and that compositions and elements may be differently positioned, or otherwise noted as in the appended claims without requirements of the written description being required thereto.

Ultraviolet radiation sun protective compositions are reported which feature micronized metal oxide inorganic particles selected from the group consisting of zinc oxide, titanium oxide, cerium oxide and mixtures thereof, the inorganic particles being coated with:
(i) a poly[C8-C20 hydroxycarboxylic acid], the coated particles measured at a 10% loading in dodecane and at 1 minute elapsed time having a Zeta Potential ranging from 2 to 10 mv, particularly from 2 to 5 mv, amounts of the poly[C8-C20 hydroxycarboxylic acid] to the inorganic particles being in a relative weight ratio of 1:100 to 1:10; or
(ii) an adduct of a hydroxy cinnamate and a silanol, amounts of the adduct to the inorganic particles being in a relative weight ratio of 1:200 to 1:4; or
(iii) mixtures of (i) and (ii).

Coatings Based on (i) Poly[C8-C20 hydroxycarboxylic acid]

The SPF of micronized metal oxide inorganic particles can be improved by oil dispersant-free coating of the metal oxide particles with poly[C8-C20 hydroxycarboxylic acid], hereinafter also referred to as PHA. Effective oil dispersant-free coating methods include (1) high speed milling, (2) liquid carbon dioxide and supercritical carbon dioxide processing and (3) solvent slurry application with subsequent solvent removal. All three methods provided coated particles with SPF values approximately double those achieved with the known oil dispersant poly[C8-C20 hydroxycarboxylic acid] coating technique.

Relative weight ratio of the poly[C8-C20 hydroxycarboxylic acid] coating to the metal oxide may range from 1:100 to 1:10, especially from 1:50 to 1:20, more especially from 1:30 to 1:25 by weight. When placed within a cosmetic product having a carrier, the relative weight ratio of the coated ultraviolet radiation sun protective compositions to the carrier may range from 1:100 to 1:4, especially from 1:50 to 1:1.5.

Viscosities of the coated particles are measured in context of a 1:1 weight ratio slurry in Tricaprylin. The Tricaprylin is a glycerol trioctanoate available from Axona Inc. under the trademark Axona®, from Sigma-Aldrich division of Merck AG, and from Abitec Inc. IUPAC name is 2,3-di(octanoyloxy)propyl octanoate. Viscosities may be taken using a Brookfield Viscometer DV-E with spindle number 4 at 23-28° C. The viscosities range from 20 to 200 cps, and particularly from 50 to 150 cps.

Advantageously the coated particles can be dosed to cosmetic product formulations in a fluid transport medium. Suitable media include triglyceride oils, hydrocarbons, silicones, fatty acids, fatty alcohols and combinations thereof. Especially useful as a fluid transport medium is Tricaprylin. When a fluid transport medium is used, the weight ratio relative to the coated particles may range from 5:1 to 1:5, especially from 2:1 to 1:2, and most especially about 1:1.

Poly[C8-C20 hydroxycarboxylic acid] are oligomers of hydroxy fatty acids. Representative oligomers are polyhydroxystearic acid (PHSA), polyricinoleic acid and mixtures thereof. Polyhydroxystearic acids are oligomers of 12-hydroxystearic acid. These form by homopolymeric condensation of 12-hydroxystearic acid monomer units. The oligomer may have from 2 to 10, preferably from 2 to 4 repeating monomer units. The material is available from Innospec Inc.

In most instances, the poly[C8-C20 hydroxycarboxylic acid] will be the one and only coating surrounding the micronized metal oxide particles. Normally, no other substance will intervene between the poly[C8-C20 hydroxycarboxylic acid] coating and the micronized metal oxide particles.

Coatings Based on (ii) an Adduct of a Hydroxy Cinnamate and a Silanol

A first component of the adduct is a hydroxy cinnamate in acid or ester form. The ester may be a C1-C8 alkyl ester selected from the group consisting of methyl, ethyl, propyl, isopropyl, phenyl and mixtures thereof. Most useful ester cinnamates are ethyl ferulate, methyl ferulate, caffeic acid methyl ester, caffeic acid phenethyl ester and caffeic acid ethyl ester. The preferred acids are ferulic acid, chlorogenic acid and caffeic acid. Precursor materials to the hydroxy cinnamate such as 7-hydroxycoumarin may also be effective.

A second component of the adduct is a silanol. Suitable silanols include those with C1-C20 alkyl and/or phenyl units which units may number from one to three, but advantageously can be a single alkyl or phenyl radical. The term ‘alkyl’ and ‘phenyl’ may include radicals with one or more hydroxyl, methoxy, ethoxy, propoxy and combination thereof substituents. Suitable but not limiting C1-C20 alkyls may be selected from the group consisting of methyl, ethyl, isopropyl, hexyl, heptyl, octyl, decyl, lauryl, monohydroxyoctyl, dihydroxyoctyl, C1-20 acyl and combinations thereof. Most preferred is the acyl radical octanoyl (also known as capryl).

The silanol may also have from one to three hydroxyl substituents. Advantageously, the silanol may have three hydroxyls forming a silane triol. Silanols may be generated by hydrolysis of silane alkoxy groups. For instance, triethoxycaprylylsilane upon de-ethoxylation/hydrolysis generates caprylylsilanetriol.

Silsesquioxanes may be suitable as a silanol. Silsesquioxanes are organosilicon compounds with the formula [RSiO3/2]n wherein R═H, alkyl, aryl or alkoxy). These silanols adopt cage-like or polymeric structures with Si—O—Si linkages and tetrahedral Si vertices. Silsesquioxanes are members of polyoctahedral silsesquioxanes (“POSS”), an example of which is TrisilanolPhenyl POSS.

Relative weight ratio of the hydroxy cinnamate to the silanol may range from 1:20 to 20:1, especially from 1:5 to 5:1, more especially from 1:2 to 2:1, and particularly 1:1

Metal Oxide Inorganic Particles

Micronized zinc oxide, titanium oxide and mixtures thereof are the most suitable metal oxides. The term ‘micronized’ means metal oxides having a primary particles size ranging from 5 to 500 nm, especially from 10 to 300 nm, when the particles are spherical or granular or amorphous. If the particles are acicular, the primary particle size may range from 5 to 50 nm by 50 to 150 nm. Primary particle size may be analyzed using Transmission Electron Microscopy.

Cosmetic Products

Cosmetic products formulated with the improved coated metal oxide sun protective particles usually include a physiologically acceptable carrier. Amounts of the carrier may range from 1 to 99.9%, preferably from 70 to 95%, optimally from 80 to 90% by weight of the product. Among the useful carriers are water, oils/emollients, gels, fatty acids, fatty alcohols, humectants, thickeners and combinations thereof. The carrier may be aqueous, anhydrous or an emulsion. Preferably the compositions are aqueous, especially water and oil emulsions of the W/O or O/W or triplex W/O/W variety. Water when present may be in amounts ranging from 5 to 98%, preferably 20 to 70%, optimally from 35 to 60% by weight.

Water when present as carrier or otherwise may advantageously be incorporated into the compositions as a deionized, sterilized or pasteurized liquid or can be heat treated or irradiated after having been mixed with other components of the composition. These treatments insure elimination of pathogenic microbes.

Oils/Emollients

Oils/Emollients may serve as dermatologically acceptable carriers. These may be in the form of silicone oils, synthetic or natural esters and hydrocarbons. Amounts of the emollients may range anywhere from 0.1 to 95%, preferably between 1 and 50% by weight of the cosmetic product.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes with viscosities of from about 5×10˜6 to 0.1 m2/s at 25° C. Among the preferred nonvolatile emollients useful in the present compositions are the polydimethyl siloxanes having viscosities from about 1×10˜5 to about 4×10˜4 m2/s at 25° C.

Another class of nonvolatile silicones are emulsifying and non-emulsifying silicone elastomers. Representative of this category is Dimethicone/Vinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester oils/emollients are:
a. Alkyl esters of saturated fatty acids having 10 to 24 carbon atoms. Examples thereof include behenyl neopentanoate, isononyl isonanonoate, isopropyl myristate and octyl stearate.
b. Ether-esters such as fatty acid esters of ethoxylated saturated fatty alcohols.
c. Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl gly col esters of C1-C30 alcohols.
d. Wax esters such as beeswax, spermaceti wax and tribehenin wax.
e. Sugar ester of fatty acids such as sucrose polybehenate and sucrose polycottonseedate.

Hydrocarbons which are suitable carriers include petrolatum, mineral oil, isoparaffins, and especially isohexadecane, available commercially as Permethyl 101 A from Presperse Inc.

Fatty acids having from 10 to 30 carbon atoms may also be suitable as carriers. Illustrative of this category are pelargonic, lauric, myristic, palmitic, stearic, isostearic, oleic, and behenic acids.

Fatty alcohols having from 10 to 30 carbon atoms are another useful category of carrier. Illustrative of this category are stearyl alcohol, lauryl alcohol, myristyl alcohol and cetyl alcohol.

Further oils/esters include hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, coriander oil, castor oil, tea tree oil, avocado oil, caprylic/capric acid triglycerides, for instance those sold by the company Stearineries Dubois or those sold under the names Miglyol 810, 812 and 818 by the company Dynamit Nobel, jojoba oil, shea butter oil and caprylyl glycol; synthetic esters and ethers, especially of fatty acids, for instance Purcellin oil, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate, sold especially under the trade name Eldew SL 205 by the company Ajinomoto; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam oil, or the mixture of n-undecane (C11) and of n-tridecane (C13) sold under the reference Cetiol UT by the company Cognis; fluoro oils that are partially hydrocarbon-based and/or silicone-based, for instance those described in document JP-A-2 295 912; silicone oils, for instance volatile or non-volatile polymethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes; mixtures thereof.

Additional examples include benzoic acid esters of C9-C15 alcohols, isononyl iso-nonanoate, C12-C15 alkyl benzoate, or any combinations thereof.

Specific examples of oils/emollients include cocoglyceride, cyclomethicone, dimethicone, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C9-C15 alcohols, isononyl iso-nonanoate, alkanes, mineral oil, silicone, dimethyl polysiloxane, ether, polyoxypropylene butyl ether, polyoxypropylene cetyl ether, C12-C15 alkyl benzoate, aryl alkyl benzoate, Isopropyl Lauroyl sarcosinate, and any combinations thereof.

Examples of amphiphilic organic solvents include: polypropylene glycol (PPG) like propylene glycol alkyl ester or alkyl ether of PPG like PPG-23 oleyl ether and PPG-36 oleate.

The above lists are only examples and not limiting. The total amount of oils/emollients present in the compositions is typically about 0.1, 0.5, 1.0, or 2.5 wt. % to about 5.0, 7.5, 10.0, 15.0, 20.0, or 30 wt. % of the total weight of the composition.

Humectants

Humectants of the polyhydric alcohol-type can be employed as carriers. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol, C1-C8 alcohols, dialkyl isosorbides such as dimethyl isosorbide and mixtures thereof. Yeast extracts (Saccharomyces cerevisiae) can also deliver soothing humectancy. The amount of humectant may range anywhere from 0.5 to 50%, preferably between 1 and 15% by weight of the product.

Thickeners

Thickeners can be utilized as part of the dermatologically acceptable carriers. Typical thickeners include crosslinked acrylates (e.g., Carbopol 982®), hydrophobically-modified acrylates (e.g., Carbopol 1382®), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums may be suitable thickeners and can include guar, xanthan, sclerotium, carrageenan, pectin and combinations of these gums. Inorganics may also be utilized as thickeners, particularly clays such as bentonites and hectorites, fumed silicas, and silicates such as magnesium aluminum silicate (Veegum®). Amounts of the thickener may range from 0.0001 to 10%, usually from 0.001 to 1%, optimally from 0.01 to 0.5% by weight of the product.

Organic UV Filters

In addition to micronized coated metal oxide inorganic particles, there may be present organic UV filters. They include both UVA and UVB protective ranges. Organic sunscreens will have at least one chromophoric group absorbing within the ultraviolet ranging from 290 to 400 nm. Chromophoric organic sunscreens may be divided into the following categories (with specific examples) including: p-Aminobenzoic acid, its salts and its derivatives (ethyl, isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Hydrocarbons (diphenylbutadiene, stilbene); Dibenzalacetone and benzalacetophenone; Naphtholsulfonates (sodium salts of 2-naphthol-3,6- disulfonic and of 2-naphthol-6,8-disulfonic acids); Dihydroxynaphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzone, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone; 4-Isopropyldibenzoylmethane; Butylmethoxydibenzoylmethane; Etocrylene; and 4-isopropyl-dibenzoylmethane).

Particularly important UV filters are: 2-ethylhexyl p-methoxycinnamate (available as Parsol MCX®), 4,4′-t-butyl methoxydibenzoylmethane (known commonly as Avobenzone, available as Parsol 1789®), octylsalicylate (available as Dermablock OS®), tetraphthalylidene dicamphor sulfonic acid (available as Mexoryl SX®), benzophenone-3 (Oxybenzone) and mixtures.

One particular embodiment utilizes a combination of UV filters that include octocrylene, avobenzone, octisalate, and homosalate, and optionally oxybenzone; wherein the ratio of each filter relative to avobenzone is as follows:
the ratio of octocrylene to avobenzone is 1.6:1.0 to 2.4:1.0;
the ratio of oxybenzone to avobenzone 0.0:1.0 to 0.016:1.0;
the ratio of octisalate to avobenzone is 1.3:1.0 to 2.0:1.0; and
the ratio of homosalate to avobenzone is 2.3:1.0 to 3.6:1.

Film Formers

Film-formers are often incorporated into sunscreen containing cosmetic compositions to ensure even coverage of the UV filters and can be used to render the composition water resistant. The film former is typically a hydrophobic material that imparts film forming and/or waterproofing characteristics. One such agent is polyethylene, which is available from New Phase Technologies as Performalene® 400, a polyethylene having a molecular weight of 400. Another suitable film former is polyethylene 2000 (molecular weight of 2000), which is available from New Phase Technologies as Performalene®. Yet, another suitable film former is synthetic wax, also available from New Phase Technologies as Performa® V-825. Other typical film-formers include acrylates/acrylamide copolymer, acrylates copolymer, acrylates/C12-C22 alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, and mixtures thereof. In some cases, the film former is acrylates/C12-C22 alkylmethacrylate copolymer sold under the tradename Allianz OPT® by ISP.

Many of the common film-forming polymers included in sunscreen containing compositions are not soluble in ethanol (such as PVP/Eicosene copolymer). A common film-former employed in ethanol-based sunscreen products is Dermacryl LT or Dermacryl 79 marketed by Akzo Nobel (INCI Name: acrylates/octylacrylamide copolymner). Dermacryl LT (CAS Number: 80570-62-3) is a hydrophobic, high molecular weight carboxylated acrylic copolymer. It functions as a film-former in a broad range of cosmetic formulations, imparting waterproofing, increased occlusivity and decreased rub-off of actives.

The above lists are only examples and not limiting.

The total amount of film-formers present in the compositions is typically in an amount of about 0.1, 0.5, 1.0, or 5 wt. % to about 5, 10, 20, or 25 wt. %, based on the total weight of the composition.

Emulsifiers/Surfactants

The cosmetic compositions typically include at least one emulsifier/surfactant, and usually more than two. They can be categorized as either amphoteric, anionic, cationic or nonionic emulsifier, used alone or as a mixture, and optionally a co-emulsifier. The emulsifiers are chosen in an appropriate manner according to the emulsion to be obtained (W/O or O/W). The emulsifier and the co-emulsifier are generally present in the composition in a proportion ranging from 0.3 wt. % to 30 M. % and preferably from 0.5 wt. % to 20 wt. % by relative to the total weight of the composition.

For W/O emulsions, examples of emulsifiers that may be mentioned include dimethicone copolyols, such as the mixture of cyclomethicone and dimethicone copolyol sold under the trade name DC 5225 C by the company Dow Corning, and alkyl dimethicone copolyols such as the lauryl dimethicone copolyol sold under the name Dow Corning 5200 Formulation Aid by the company Dow Corning, and the cetyl dimethicone copolyol sold under the name Abil EM 90™ by the company Goldschmidt. A crosslinked elastomeric solid organopolysiloxane comprising at least one oxyalkylene group, such as those obtained according to the procedure of Examples 3, 4 and 8 of U.S. Pat. No. 5,412,004 and of the examples of U.S. Pat. No. 5,811,487, especially the product of Example 3 (synthesis example) of U.S. Pat. No. 5,412,004, such as the product sold under the reference KSG 21 by the company Shin-Etsu, may also be used as surfactants for W/O emulsions.

For O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan: oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof.

The fatty acid esters of a sugar that can be used as nonionic amphiphilic lipids can be chosen in particular from the group comprising esters or mixtures of esters of a C8-C22 fatty acid and of sucrose, of maltose, of glucose or of fructose, and esters or mixtures of esters of a C14-C22 fatty acid and of methylglucose. The C8-C22 or C14-C22 fatty acids forming the fatty unit of the esters that can be used in the emulsion comprise a saturated or unsaturated linear alkyl chain having, respectively, from 8 to 22 or from 14 to 22 carbon atoms. The fatty unit of the esters can be chosen in particular from stearates, behenates, arachidonates, palmitates, myristates, laurates, caprates and mixtures thereof.

By way of example of esters or of mixtures of esters of a fatty acid and of sucrose, of maltose, of glucose or of fructose, mention may be made of sucrose monostearte, sucrose distearate, sucrose tristearate and mixtures thereof, such as the products sold by the company Croda under the name Crodesta F50, F70, F110 and F160 having, respectively, an HLB (Hydrophilic Lipophilic Balance) of 5, 7, 11 and 16; and, by way of example of esters or of mixtures of esters of a fatty acid and of methylglucose, mention may be made of the distearate of methylglucose and of polyglycerol-3, sold by the company Goldschmidt under the name Tego-care 450. Mention may also be made of glucose monoesters or maltose monoesters, such as methyl O-hexadecanoyl-6-D-glucoside and O-hexadecanoyl-6-D-maltoside.

The fatty alcohol ethers of a sugar that can be used as nonionic amphiphilic lipids can be chosen in particular from the group comprising ethers or mixtures of ethers of a C8-C22 fatty alcohol and of glucose, of maltose, of sucrose or of fructose, and ethers or mixtures of ethers of a C14-C22 fatty alcohol and of methylglucose. They are in particular alkylpolyglucosides.

The C8-C22 or C14-C22 fatty alcohols forming the fatty unit of the ethers that can be used in the emulsion of the present disclosure comprise a saturated or unsaturated linear alkyl chain having, respectively, from 8 to 22 or from 14 to 22 carbon atoms. The fatty unit of the ethers can be chosen in particular from decyl, cetyl, behenyl, arachidyl, stearyl, palmityl, myristyl, lauryl, capryl and hexadecanoyl units, and mixtures thereof such as cetearyl.

By way of example of fatty alcohol ethers of a sugar, mention may be made of alkylpolyglucosides, such as decylglucoside and laurylglucoside sold, for example, by the company Henkel under the respective names Plantaren 2000 and Plantaren 1200, cetostearylglucoside, optionally as a mixture with cetostearyl alcohol, sold, for example, under the name Montanov 68 by the company Seppic, under the name Tego-care CG90 by the company Goldschmidt and under the name Emulgade KE3302 by the company Henkel, and also arachidylglucoside, for example in the form of the mixture of arachidyl and behenyl alcohols and of arachidylglucoside sold under the name Montanov 202 by the company Seppic. Nonionic amphiphilic lipid of this type particularly useful are sucrose monostearate, sucrose distearate, sucrose tristearate and mixtures thereof, and the distearate of methylglucose and of polyglycerol-3, and alkylpolyglucosides.

The glycerol fatty esters that can be used as nonionic amphiphilic lipids can be chosen in particular from the group comprising the esters formed from at least one acid comprising a saturated linear alkyl chain having from 16 to 22 carbon atoms, and from 1 to 10 glycerol units. Use may be made of one or more of these glycerol fatty esters in the emulsions of the instant disclosure.

By way of example of a surfactant that can be used in the emulsion of the instant disclosure, mention may be made of decaglycerol monostearate, distearate, tristearate and pentastearate (10 glycerol units) (CTFA names: polyglyceryl-10 stearate, polyglyceryl-10 distearate, polyglyceryl-10 tristearate, polyglyceryl-10 pentastearate), such as the products sold under the respective names Nikkol Decaglyn 1-S, 2-S, 3-S and 5-S by the company Nikko, and diglyceryl monostearate (CTFA name: polyglyceryl-2 stearate) such as the product sold by the company Nikko under the name Nikkol DGMS.

The sorbitan fatty esters that can be used as nonionic amphiphilic lipids chosen in particular from the group comprising esters of a C16-C22 fatty acid and of sorbitan and oxyethylenated esters of a C16-C22 fatty acid and of sorbitan. They are formed from at least one fatty acid comprising at least one saturated linear alkyl chain, having, respectively, from 16 to 22 carbon atoms, and from sorbitol or from ethoxylated sorbitol. The oxyethylenated esters generally comprise from 1 to 100 ethylene oxide units, and preferably from 2 to 40 ethylene oxide (EO) units.

These esters can be chosen in particular from stearates, behenates, arachidates, palmitates and mixtures thereof. Stearates and palmitates are preferably used.

By way of example of sorbitan fatty ester and of an oxyethylenated sorbitan fatty ester, mention may be made of sorbitan monostearate (CTFA name: sorbitan stearate) sold by the company ICI under the name Span 60, sorbitan monopalmitate (CTFA name: sorbitan palmitate) sold by the company ICI under the name Span 40, or sorbitan 20 EO tristearate (CTFA name: polysorbate 65) sold by the company ICI under the name Tween 65.

The ethoxylated fatty ethers are typically ethers made up of 1 to 100 ethylene oxide units and of at least one fatty alcohol chain having from 16 to 22 carbon atoms. The fatty chain of the ethers can be chosen in particular from behenyl, arachidyl, stearyl and cetyl units, and mixtures thereof, such as cetearyl. By way of example of ethoxylated fatty ethers, mention may be made of ethers of behenyl alcohol comprising 5, 10, 20 and 30 ethylene oxide units (CTFA names: beheneth-5, beheneth-10, beheneth-20 and beheneth-30), such as the products sold under the names Nikkol BBS, BB10, BB20 and BB30 by the company Nikko, and the ether of stearyl alcohol comprising 2 ethylene oxide units (CTFA name: steareth-2), such as the product sold under the name Brij 72 by the company ICI.

The ethoxylated fatty esters that can be used as nonionic amphiphilic lipids are esters made up of 1 to 100 ethylene oxide units and of at least one fatty acid chain comprising from 16 to 22 carbon atoms. The fatty chain of the esters can be chosen in particular from stearate, behenate, arachidate and palmitate units, and mixtures thereof. By way of example of ethoxylated fatty esters, mention may be made of the ester of stearic acid comprising 40 ethylene oxide units, such as the product sold under the name Myrj 52 (CTFA name: PEG-40 stearate) by the company ICI, and the ester of behenic acid comprising 8 ethylene oxide units (CTFA name: PEG-8 behenate), such as the product sold under the name Compritol HD5 ATO by the company Gattefosse.

The block copolymers of ethylene oxide and of propylene oxide that can be used as nonionic amphiphilic can be chosen in particular from poloxamers and in particular from Poloxamer 231, such as the product sold by the company ICI under the name Pluronic L81 of formula (V) with x=z=6, y=39 (HLB 2); Poloxamer 282, such as the product sold by the company ICI under the name Pluronic L92 of formula (V) with x=z=10, y=47 (HLB 6); and Poloxamer 124, such as the product sold by the company ICI under the name Pluronic L44 of formula (V) with x=z=11, y=21 (HLB 16).

As nonionic amphiphilic lipids, mention may also be made of the mixtures of nonionic surfactants described in document EP-A-705593, incorporated herein for reference.

Suitable hydrophobically-modified emulsifiers include, for example, inulin lauryl carbamate, commercially available from Beneo Orafti under the tradename Inutec SPI.

The above lists are only examples and not limiting.

The total amount of emulsifier present in the compositions is typically in an amount of about 0.1, 0.2, or 0.5 wt. % to about 4.0, 5.0, 6.0, or 7.5 wt. %, based on the total weight of the composition.

Gelling Agents

Gelling agents may also be included in the sunscreen containing compositions.

Examples of suitable hydrophilic gelling agents include carboxyvinyl polymers such as the Carbopol products (carbomers) and the Pemulen products (acrylate/C10-C30-alkylacrylate copolymer); polyacrylamides, for instance the crosslinked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymerfisohexadecane/polysorbate 80) by the company SEPPIC; 2-acrylamido-2-methylpropanesulfonic acid polymers and copolymers, which are optionally crosslinked and/or neutralized, for instance the poly(2-acrylamido-2-methylpropanesulfonic acid) sold by the company Hoechst under the trade name “Hostacerin AMPS” (CTFA name: ammonium polyacryldimethyltauramide); cellulose-based derivatives such as hydroxyethylcellulose; polysaccharides and especially gums such as xanthan gum; and mixtures thereof.

Lipophilic gelling agents (thickeners) that may be mentioned include modified clays such as hectorite and its derivatives, for instance the products sold under the name bentone.

In some instances, the gelling agent is ammonium acryloyldimethyltaurate/steareth-25 methacrylate crosspolymer, commercially available from Clariant under the tradename Aristoflex HMS.

The above lists are only examples and not limiting.

The gelling agent is typically used in an amount of about 0.05 to about 1.5% by weight, from about 0.08 to about 1.0% by weight, or about 0.1 to about 0.5% by weight, based on the total weight of the composition.

The above lists are only examples and not limiting.

Skin Lightening and Antiaging Agents

Cosmetic products intended to be skin lighteners normally will be formulated with a skin lightening compound. Illustrative substances are placental extract, lactic acid, niacinamide, arbutin, kojic acid, ferulic acid, hydroquinone, resorcinol and derivatives including 4-substituted resorcinols, phenylethyl resorcinol and combinations thereof. Amounts of these substances may range from 0.1 to 10%, preferably from 0.5 to 2% by weight of the product.

Also included may be such materials as turmeric (curcumin), ubiquinone (Co-Enzyme CoQ10), resveratrol, alpha-lipoic acid, ellagic acid, kinetin, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B, Ceramide 6 and Ceramide 7) as well as pseudoceramides are useful. Amounts of these materials may range from 0.000001 to 10%, preferably from 0.0001 to 1% by weight of the composition.

Vitamins and Flavonoids

Cosmetic compositions may include vitamins. Illustrative vitamins are Vitamin A (retinol), Vitamin B2, Vitamin B3 (niacinamide), Vitamin B5 (pantothenic acid), Vitamin B6, Vitamin B12, Vitamin C, Vitamin D, Vitamin E, Vitamin K and Biotin. Derivatives of the vitamins may also be employed. For instance, Vitamin C derivatives include ascorbyl tetraisopalmitate, magnesium ascorbyl phosphate and ascorbyl glycoside (Vitamin CG). Derivatives of Vitamin E include tocopheryl acetate, tocopheryl palmitate and tocopheryl linoleate. DL-panthenol and derivatives may also be employed. A particularly suitable Vitamin B6 derivative is Pyridoxine Palmitate. Flavonoids may also be useful, particularly glucosyl hesperidin, rutin, and soy isoflavones (including genistein, daidzein, equol, and their glucosyl derivatives and mixtures thereof. Also useful are carotenoids such as lycopene (available from watermelon extracts).

Total amount of vitamins or flavonoids when present may range from 0.0001 to 10% by weight of the composition.

The cosmetic compositions may be formulated into a wide variety of product types that include but are not limited to solutions, suspensions, lotions, creams, gels, toners, sticks, sprays, ointments, cleansing liquid washes and solid bars, shampoos and hair conditioners, hair colorants, pastes, foams, powders, mousses, wipes, hydrogels, film-forming products, facial and skin masks, make-up such as foundations, lipsticks, eye liners, eye shadows, and mascara.

Cationic Polymers

Cationic polymers may be useful in certain cosmetic products. Illustrative are polyquatemium 4. polyquaternium 6, polyquaternium 7, polyquaternium 10, polyquaternium 11, polyquaternium 16, polyquaternium 22, and polyquaternium 32. Cationic polymers useful in the present invention include, but are not limited to, polyquaternium 4. polyquaternium 6, polyquaternium 7, polyquaternium 10, polyquaternium 11, polyquaternium 16, polyquaternium 22, polyquaternium 28, polyquaternium 32, and guar hydroxypropyltrimonium chloride. Preferred cationic polymers include POLYMER JR-125, POLYMER JR-400, Polymer JR-30M hydroxyethyl cellulosic polymers (polyguaternium 10) available from AMERCHOL: JAGUAR C13-S, guar hydroxypropyltrimonium chloride, available from Rhodia; and MERQUAT 100 and 280, a dimethyl dialkyl ammonium chloride (polyduaternium 6) available from Nalco. The cationic polymer when present may be in an amount of from greater than 0% to about 15%, preferably from about 0.5 to about 10% by weight, and more preferably from about 1 to about 5% by weight, based on the total weight of the composition.

Preservatives

Preservatives may be incorporated into the cosmetic compositions to protect against the growth of potentially harmful microorganisms. Suitable traditional preservatives are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are methylchloroisothiazolinone and methyl isothiazolinone combinations, phenoxyethanol, methyl paraben, propyl paraben, urea, imidazolidinyl urea, sodium dehydroacetate and benzyl alcohol. Preservatives may be employed in amounts ranging from 0.01%> to 2% by weight of the cosmetic composition.

Desquamation Agents

Desquamation agents may be present. Illustrative are the monocarboxylic acids. Monocarboxylic acids may be substituted or unsubstituted with a carbon chain length of up to 16. Particularly preferred carboxylic acids are the alpha-hydroxycarboxylic acids, beta-hydroxycarboxylic or polyhydroxycarboxylic acids. The term “acid” is meant to include not only the free acid but also salts and C(—C3o alkyl or aryl esters thereof and lactones generated from removal of water to form cyclic or linear lactone structures. Representative acids are glycolic, lactic, malic and tartaric acids. A representative salt that is particularly preferred is ammonium lactate. Salicylic acid is representative of the beta-hydroxycarboxylic acids. Amounts of these materials when present may range from 0.01 to 15% by weight of the cosmetic composition.

Preferred desquamation agents may be selected from the group consisting of glycolic acid, lactic acid, salicylic acid, retinoic acid, retinol and mixtures thereof, and including salt forms thereof

Colorants

Colorants may either be dyes or pigments. A distinction is usually made between a pigment, which is insoluble in its vehicle (resulting in a suspension), and a dye, which either is itself a liquid or is soluble in its vehicle (resulting in a solution). A colorant can act as either a pigment or a dye depending on the vehicle involved. In some cases, a pigment can be manufactured from a dye by precipitating a soluble dye with a metallic salt. The resulting pigment is called a lake pigment.

Among the more common dyes are Alizarin, Azophloxin, Chrysoidin, Congo Red, Fuchsin acid, Gentian violet, Janus green, Methyl Red, Naphthol Green, Naphthol Yellow, Rose Bengal, Sudan II, Titan Yellow and combinations thereof. Amongst pigments, titanium dioxide and aluminum lakes (aluminum salts of dyes) are most common. Amounts of the colorant may, according to the type of cosmetic product (lipstick, foundation, hair dye, etc) range from 0.000001 to 10%, usually from 0.01 to 5% by weight of the cosmetic composition.

The sun protection factor (SPF rating) has been used to qualitatively describe differences in protective efficacy. SPF is a measure of the fraction of sunburn-producing UV rays that reach the skin. For example, “SPF 15” means that 1/15th of the burning radiation will reach the skin, assuming sunscreen is applied evenly at a thick dosage of 2 milligrams per square centimeter (mg/cm2). A user can determine the effectiveness of a sunscreen by multiplying the SPF factor by the length of time it takes for him or her to suffer a burn without sunscreen. Thus, if a person develops a sunburn in 10 minutes when not wearing a sunscreen, the same person in the same intensity of sunlight will avoid sunburn for 150 minutes if wearing a sunscreen with an SPF of 15.

Besides an in vivo measurement, SPF can also be measured in vitro with the help of a specially designed spectrometer. In this case, the actual transmittance of the sunscreen is measured, along with the degradation of the product due to being exposed to sunlight. Transmittance of the sunscreen must be measured over all wavelengths in sunlight's UVB-UVA range (290-400 nm), along with a table of how effective various wavelengths are in causing sunburn (the erythema! action spectrum) and the standard intensity spectrum of sunlight. Evaluations of SPF in the Examples which follow report results by the in vitro method.

The compositions according to the instant disclosure may be prepared according to techniques that are well known to those skilled in the art, in particular those intended for the preparation of emulsions of oil-in-water or water-in-oil type. They may be in particular in the form of a simple or complex emulsion (O/W, W/O, O/W/O or W/O/W emulsion) such as a cream or a milk, in the form of a gel or a cream-gel, or in the form of a lotion.

The present disclosure is also directed to methods of protecting a keratinous substrate from ultraviolet radiation and to methods of absorbing ultraviolet light. Such methods encompass applying a sunscreen composition to a keratinous substrate and subjecting the keratinous substrate to ultraviolet radiation.

Where the following terms are used in this specification, they are used as defined below. The terms “comprising,” “having,” and “including” are used in their open, non-limiting sense. The terms “a” and “the” are understood to encompass the plural as well as the singular. As used herein, the expression “at least one” means one or more and thus includes individual components as well as mixtures/combinations. “Cosmetically acceptable” means that the item in question is compatible with any keratinous substrate. For example, “cosmetically acceptable carrier” means a carrier that is compatible with any keratinous substrate (e.g., hair or scalp). A “physiologically acceptable medium” means a medium which is not toxic and can be applied to the skin, lips, hair, scalp, lashes, brows, nails or any other cutaneous region of the body. The composition of the instant disclosure may especially constitute a cosmetic or dermatological composition. The phrase “essentially without” refers to less than or equal to 0.5, 0.1, 0.05 or 0.01 wt. %. The phrase “stable emulsion” refers to a composition that does not undergo phase separation at a temperature of 45 C°.

Except in the examples, or where otherwise explicitly indicated, all numbers in this description indicating amounts of material or conditions of reaction, physical properties of materials and/or use are to be understood as modified by the word “about.” All amounts are by weight of the compositions, unless otherwise specified.

It should be noted that in specifying any range of concentration or amount, any particular upper concentration can be associated with any particular lower concentration or amount. The disclosure of the invention as found herein is to be considered to cover all embodiments as found in the claims as being multiply dependent upon each other irrespective of the fact that claims may be found without multiple dependency or redundancy.

“Product” as used herein, is meant to include a formulated cosmetic sunscreen containing composition for topical application to skin or hair of mammals, especially humans or for deposition onto textiles via laundering.

The instant disclosure will be better understood from the examples that follow, all of which are intended for illustrative purposes only and are not meant to limit the scope of the instant disclosure in any way.

Various operations may be described as multiple discrete operations in turn, in a manner that may be helpful in understanding embodiments of the present invention; however, the order of description should not be construed to imply that these operations are order dependent.

Although only a few embodiments have been disclosed in detail above, other embodiments are possible, and the inventors intend these to be encompassed within this specification. The specification describes certain technological solutions to solve the technical problems that are described either expressly or inherently in this application. This disclosure describes alternative and adaptive embodiments, with any ranges, methods of producing or providing, including any listed herewith, and the claims are intended to cover any modification or alternative or generalization of these embodiments which might be predictable to a person having ordinary skill in the art.

### EXAMPLES

Set A.

PICKERING EMULSION

| Phase A | Ingredients | INCI Name | Supplier | 1 | 2 | 3 | 4 | 5 |
|---|---|---|---|---|---|---|---|---|
| A | Water | Water | Any | 60.0 | 60.0 | 60.0 | 60.0 | 60.0 |
| B | Veegum Ultra Granules | Magnesium Aluminium | Veegum ® Ultra | 2.0 | 2.0 | 2.0 | 2.0 | 2.0 |
| | | Silicate | | | | | | |
| | Frametime CX | Bentonite & Xanthan | EPHYLA | 3.0 | 3.0 | 3.0 | 3.0 | 3.0 |
| | | gum & Citric Acid | | | | | | |
| | Glycerin | Glycerine | Any | 6.0 | 6.0 | 6.0 | 6.0 | 6.0 |
| C | Sodium Chloride | Sodium Chloride | Any | 1.0 | 1.0 | 1.0 | 1.0 | 1.0 |
| | Zemea Propanediol | Propanediol | Dupont | 3.0 | 3.0 | 3.0 | 3.0 | 3.0 |
| D | Desert Date | Balanites Roxburghii | EPHYLA/NSL | 2.0 | 2.0 | 2.0 | 2.0 | 2.0 |
| | | Seed Oil | | | | | | |
| | HTRE | Helianthus Annuus Seed | Ephyla/nsl | 2.0 | 2.0 | 2.0 | 2.0 | 2.0 |
| | NS EX-81 | Polyglyceryl-8 Oleate | Next Step Lab | 1.0 | 1.0 | 1.0 | 1.0 | 1.0 |
| E | Lanette 22 | Behenyl Alcohol | BASF | 2.0 | 2.0 | 2.0 | 2.0 | 2.0 |
| C | SuperZinc ® 1000 | Zinc Oxide, Ethyl Ferulate, | Vizor | 15.0 | 0.0 | 15.0 | 0.0 | 0.0 |
| | | Triethoxycaprylyl Silane | | | | | | |
| | SuperZinc Natural | Zinco Oxide, | Vizor | 0.0 | 15.0 | 0.0 | 15.0 | 15.0 |
| | | Polyhydroxystearic Acid | | | | | | |
| | Super Ti 1000 | Titanium Dioxide, Ethyl | Vizor | 2.0 | 0.0 | 0.0 | 2.0 | 0.0 |
| | | Ferulate, Triethoxycapryl | | | | | | |
| | | Silane | | | | | | |
| | Super Ti Natural | Titanium Dioxide, | Vizor | 0.0 | 2.0 | 2.0 | 0.0 | 0.0 |
| | | Polyhydroxystearic Acid | | | | | | |
| | MT-500 | Titanium Dioxide | Presperse | 0.0 | 0.0 | 0.0 | 0.0 | 2.0 |
| F | Phenoxyethanol | Phenoxyethanol | Any | 1.0. | 1.0 | 1.0 | 1.0 | 1.0 |
| | | | | 100 | 100 | 100 | 100 | 100 |

### Procedure:

1. Combine Phase A in a beaker and set aside.
2. In a separate beaker combine Phase B and hand mix until frametime is completely dispersed.
3. Add Phase B to A and homogenize until fully dispersed
4. Add Phase C to Phase AB and continue homomixing. Heat to 60-70° C.
5. Combine Phase D in sequential order while homomixing for 30-40 mins until zinc is fully dispersed
6. Heat Phase D to 75-80 C and add Phase E while homoxing
7. Add Phase DE to Phase ABC, remove from heat, and continue homogenizing
8. Add Phase F at 50 C and homomix for 3 minutes.
9. Transfer batch to a sweep mixer at 40° C. and continue mixing.
10. Drop batch at 30° C.

Set B.

Fomula: NSL21-61
Product: Super Thin SPF 50

| Phase | Tradename | Description | Supplier | 1% | 2% | 3% | 4% | 5% |
|---|---|---|---|---|---|---|---|---|
| A | NS Vsol-5 | Isododecane, Hydrogenated | Next | 12.00 | 12.00 | 12.00 | 12.00 | 12.00 |
| | | Polydecene, Dimethicone | Step Lab | | | | | |
| | Jeesile IDD | Isododecane, Dimethicone | Jeen | 6.40 | 6.40 | 6.40 | 6.40 | 6.40 |
| | | Crosspolymer-3 | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) bran extract | Next | 2.00 | 2.00 | 2.00 | 2.00 | 2.00 |
| | | | Step Lab | | | | | |
| | Caprylyl Glycol | Caprylyl Glycol | Any | 0.25 | 0.25 | 0.25 | 0.25 | 0.25 |
| | Phenoxyethanol | Phenoxyethanol | Any | 0.35 | 0.35 | 0.35 | 0.35 | 0.35 |
| B | NS Plant Gel 2002 | Dodecane (and) Stearalkonium | Next | 10.00 | 10.00 | 10.00 | 10.00 | 10.00 |
| | | Bentonite (and) Triethyl Citrate | Step Lab | | | | | |
| | Abil EM 90 | Cetyl PEG/PPG-10/1 | Evonik | 3.00 | 3.00 | 3.00 | 3.00 | 3.00 |
| | | Dimethicone | | | | | | |
| | NS Gel IDD | Isododecane(and) Quaternium- | Next | 6.40 | 6.40 | 6.40 | 6.40 | 6.40 |
| | | 90 Bentonite (and) Propylene | Step Lab | | | | | |
| | | Carbonate | | | | | | |
| C | SuperZinc ® 1000 | Zinc Oxide, Ethyl Ferulate, | Vizor | 17.00 | 0.00 | 0.00 | 0.00 | 0.00 |
| | | Triethoxycaprylyl Silane | | | | | | |
| | SuperZinc Natural | Zinco Oxide, | Vizor | 0.00 | 17.00 | 0.00 | 0.00 | 0.00 |
| | | Polyhydroxystearic Acid | | | | | | |
| | Super Ti 1000 | Titanium Dioxide, Ethyl | Vizor | 0.00 | 0.00 | 17.00 | 0.00 | 0.00 |
| | | Ferulate, Triethoxycapryl | | | | | | |
| | | Silane | | | | | | |
| | Super Ti Natural | Titanium Dioxide, | Vizor | 0.00 | 0.00 | 0.00 | 17.00 | 0.00 |
| | | Polyhydroxystearic Acid | | | | | | |
| | MT-500 | Titanium Dioxide | Presperse | 0.00 | 0.00 | 0.00 | 0.00 | 17.00 |
| | NS PGPR | Polyglyceryl 3 Polyricinoleate | Next | 0.30 | 0.30 | 0.30 | 0.30 | 0.30 |
| | | | Step Lab | | | | | |
| | NS Boost SiD | Mica, Silica, Dimethicone | Next | 3.00 | 3.00 | 3.00 | 3.00 | 3.00 |
| | | | Step Lab | | | | | |
| | NS Spherilon-10 | Nylon-12 | Next | 1.50 | 1.50 | 1.50 | 1.50 | 1.50 |
| | | | Step Lab | | | | | |
| D | Deionized Water | Water | Any | 19.00 | 19.00 | 19.00 | 19.00 | 19.00 |
| | Magnesium Sulfate | Magnesium Sulfate | Any | 1.00 | 1.00 | 1.00 | 1.00 | 1.00 |
| | NS Phytomoist | Tremella Fuciformis, Betain, | Next | 3.00 | 3.00 | 3.00 | 3.00 | 3.00 |
| | | Glycerin, Water, Potassium | Step Lab | | | | | |
| | | Sorbate, Phenoxyethanol | | | | | | |
| | | | | 100.00 | 100.00 | 100.00 | 100.00 | 100.00 |

Note:
this a W/O emulsion

### Lab Procedure

1. Combine phase A ingredients and lightning mix at 500 rpm for 5 minutes.
2. Add phase B to phase A, and lightning mix at 300-500 rpm for 15 minutes to break up agglomerates.
3. Add phase C ingredients to phases AB with homomixing at 3,000-3,500 rpm. Allow to mix for 10-15 minutes. Scrape down the sides of the beaker, and the homogenizer.
4. Combine phase D ingredient in a separate beaker, and lightning mix until uniformly dispersed.
5. Add phase D to phases A-C slowly with homomixing at 3,500 rpm-4,000 rpm; continue mixing for 15 minutes
6. Drop batch

Set C.

Formula:
Product: SPF-50

| Phase | Tradename | Description | Supplier | 1% | 2% | 3% | 4% | 5% |
|---|---|---|---|---|---|---|---|---|
| A | NS Vsol-5 | Isododecane. | Next | 12.00 | 12.00 | 12.00 | 12.00 | 12.00 |
| | | Hydrogenated Polydecene, | Step Lab | | | | | |
| | | Dimethicone | | | | | | |
| | Jeesile IDD | Isododecane, Dimethicone | Jeen | 6.40 | 6.40 | 6.40 | 6.40 | 6.40 |
| | | Crosspolymer-3 | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) bran | Next | 2.00 | 2.00 | 2.00 | 2.00 | 2.00 |
| | | extract | Step Lab | | | | | |
| | Caprylyl Glycol | Caprylyl Glycol | Any | 0.25 | 0.25 | 0.25 | 0.25 | 0.25 |
| | Phenoxyethanol | Phenoxyethanol | Any | 0.35 | 0.35 | 0.35 | 0.35 | 0.35 |
| B | NS Plant Gel 2002 | Dodecane (and) | Next | 10.00 | 10.00 | 10.00 | 10.00 | 10.00 |
| | | Stearalkonium Bentonite | Step Lab | | | | | |
| | | (and) Triethyl Citrate | | | | | | |
| | Abil EM 90 | Cetyl PEG/PPG-10/1 | Evonik | 3.00 | 3.00 | 3.00 | 3.00 | 3.00 |
| | | Dimethicone | | | | | | |
| | NS Gel IDD | Isododecane(and) | Next | 6.40 | 6.40 | 6.40 | 6.40 | 6.40 |
| | | Quaternium-90 Bentonite | Step Lab | | | | | |
| | | (and) Propylene Carbonate | | | | | | |
| C | SuperZinc ® 1000 | Zinc Oxide, Ethyl Ferulate, | Vizor | 15.00 | 0.00 | 15.00 | 0.00 | 0.00 |
| | | Triethoxycaprylyl Silane | | | | | | |
| | SuperZinc Natural | Zinco Oxide, | Vizor | 0.00 | 15.00 | 0.00 | 15.00 | 15.00 |
| | | Polyhydroxystearic Acid | | | | | | |
| | Super Ti 1000 | Titanium Dioxide, Ethyl | Vizor | 2.00 | 0.00 | 0.00 | 2.00 | 0.00 |
| | | Ferulate, Triethoxycapryl | | | | | | |
| | | Silane | | | | | | |
| | Super Ti | Titanium Dioxide, | Vizor | 0.00 | 2.00 | 2.00 | 0.00 | 0.00 |
| | Natural | Polyhydroxystearic Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step Lab | | | | | |
| | NS Boost SiD | Mica, Silica, Dimethicone | Next | 3.00 | 3.00 | 3.00 | 3.00 | 3.00 |
| | | | Step Lab | | | | | |
| | NS Spherilon-10 | Nylon-12 | Next | 1.50 | 1.50 | 1.50 | 1.50 | 1.50 |
| | | | Step Lab | | | | | |
| D | Deionized Water | Water | Any | 19.00 | 19.00 | 19.00 | 19.00 | 19.00 |
| | Magnesium Sulfate | Magnesium Sulfate | Any | 1.00 | 1.00 | 1.00 | 1.00 | 1.00 |
| | NS Phytomoist | Tremella Fuciformis, | Next | 3.00 | 3.00 | 3.00 | 3.00 | 3.00 |
| | | Betain, Glycerin, Water, | Step Lab | | | | | |
| | | Potassium Sorbate, | | | | | | |
| | | Phenoxyethanol | | | | | | |
| | | | | 100.0 | 100.0 | 100.0 | 100.0 | 100.00 |

Note:
this a W/O emulsion

### Lab Procedure

1. Combine phase A ingredients and lightning mix at 500 rpm for 5 minutes.
2. Add phase B to phase A, and lightning mix at 300-500 rpm for 15 minutes to break up agglomerates.
3. Add phase C ingredients to phases AB with homomixing at 3,000-3,500 rpm. Allow to mix for 10-15 minutes. Scrape down the sides of the beaker, and the homogenizer.
4. Combine phase D ingredient in a separate beaker, and lightning mix until uniformly dispersed.
5. Add phase D to phases A-C slowly with homomixing at 3,500 rpm-4,000 rpm; continue mixing for 15 minutes.
6. Drop batch

Set D.

Formula:
Product: SPF 50

| Phase | Tradename | Description | Supplier | 1% | 2% | 3% | 4% | 5% |
|---|---|---|---|---|---|---|---|---|
| A | Dermol IPP | Isopropyl Palmitate | Alzo | 11.00 | 11.00 | 11.00 | 11.00 | 11.00 |
| | Neobee M | Caprylic/Capric | Stepan | 6.40 | 6.40 | 6.40 | 6.40 | 6.40 |
| | | Triglyceride | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) bran | Next | 2.00 | 2.00 | 2.00 | 2.00 | 2.00 |
| | | extract | Step Lab | | | | | |
| | Caprylyl Glycol | Caprylyl Glycol | Any | 0.25 | 0.25 | 0.25 | 0.25 | 0.25 |
| | Phenoxyethanol | Phenoxyethanol | Any | 0.35 | 0.35 | 0.35 | 0.35 | 0.35 |
| B | NS Gel IDD | Quaternium-90 Bentonite, | Next | 10.00 | 10.00 | 10.00 | 10.00 | 10.00 |
| | | Isododecane, Propylene | Step Lab | | | | | |
| | | Carbonate | | | | | | |
| | KSG-210 | Dimethicone, Dimethicone- | Shin-etsu | 5.00 | 5.00 | 5.00 | 5.00 | 5.00 |
| | | 10/15 Crosspolymer | | | | | | |
| | NS Gel IDD | Isododecane(and) | Next | 6.40 | 6.40 | 6.40 | 6.40 | 6.40 |
| | | Quaternium-90 Bentonite | Step Lab | | | | | |
| | | (and) Propylene Carbonate | | | | | | |
| C | SuperZinc ® 1000 | Zinc Oxide, Ethyl Ferulate, | Vizor | 15.00 | 0.00 | 15.00 | 0.00 | 0.00 |
| | | Triethoxycaprylyl Silane | | | | | | |
| | SuperZinc Natural | Zinco Oxide, | Vizor | 0.00 | 15.00 | 0.00 | 15.00 | 15.00 |
| | | Polyhydroxystearic Acid | | | | | | |
| | Super Ti 1000 | Titanium Dioxide, Ethyl | Vizor | 2.00 | 0.00 | 0.00 | 2.00 | 0.00 |
| | | Ferulate, Triethoxycapryl | | | | | | |
| | | Silane | | | | | | |
| | Super Ti Natural | Titanium Dioxide, | Vizor | 0.00 | 2.00 | 2.00 | 0.00 | 0.00 |
| | | Polyhydroxystearic Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step Lab | | | | | |
| | NS Boost SiD | Mica, Silica, Dimethicone | Next | 3.00 | 3.00 | 3.00 | 3.00 | 3.00 |
| | | | Step Lab | | | | | |
| | NS Spherilon-10 | Nylon-12 | Next | 1.50 | 1.50 | 1.50 | 1.50 | 1.50 |
| | | | Step Lab | | | | | |
| D | Deionized Water | Water | Any | 19.00 | 19.00 | 19.00 | 19.00 | 19.00 |
| | Magnesium Sulfate | Magnesium Sulfate | Any | 1.00 | 1.00 | 1.00 | 1.00 | 1.00 |
| | NS Phytomoist | Tremella Fuciformis, | Next | 2.00 | 2.00 | 2.00 | 2.00 | 2.00 |
| | | Betain, Glycerin, Water, | Step Lab | | | | | |
| | | Potassium Sorbate, | | | | | | |
| | | Phenoxyethanol | | | | | | |
| | | | | 100.0 | 100.0 | 100.0 | 100.0 | 100.0 |

Note:
this a W/O emulsion

### Lab Procedure

1. Combine phase A ingredients and lightning mix at 500 rpm for 5 minutes.
2. Add phase B to phase A, and lightning mix at 300-500 rpm for 15 minutes to break up agglomerates
3. Add phase C ingredients to phases AB with homomixing at 3,000-3,500 rpm. Allow to mix for 10-15 minutes. Scrape down the sides of the beaker, and the homogenizer.
4. Combine phase D ingredient in a separate beaker, and lightning mix until uniformly dispersed.
5. Add phase D to phases A-C slowly with homomixing at 3,500 rpm-4,000 rpm; continue mixing for 15 minutes.
6. Drop batch

Set E.

Formula:
Product: SPF 50

| Phase | Tradename | Description | Supplier | 1% | 2% | 3% | 4% | 5% |
|---|---|---|---|---|---|---|---|---|
| A | Dermol IPP | Isopropyl Palmitate | Alzo | 11.00 | 11.00 | 11.00 | 11.00 | 11.00 |
| | Neobee M | Caprylic/Capric | Stepan | 6.40 | 6.40 | 6.40 | 6.40 | 6.40 |
| | | Triglyceride | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) bran | Next | 2.00 | 2.00 | 2.00 | 2.00 | 2.00 |
| | | extract | Step Lab | | | | | |
| | Caprylyl Glycol | Caprylyl Glycol | Any | 0.25 | 0.25 | 0.25 | 0.25 | 0.25 |
| | Phenoxyethanol | Phenoxyethanol | Any | 0.35 | 0.35 | 0.35 | 0.35 | 0.35 |
| B | NS Gel IDD | Quaternium-90 Bentonite, | Next | 10.00 | 10.00 | 10.00 | 10.00 | 10.00 |
| | | Isododecane, Propylene | Step Lab | | | | | |
| | | Carbonate | | | | | | |
| | KSG-210 | Dimethicone, Dimethicone- | Shin-etsu | 5.00 | 5.00 | 5.00 | 5.00 | 5.00 |
| | | 10/15 Crosspolymer | | | | | | |
| | NS Gel IDD | Isododecane(and) | Next | 6.40 | 6.40 | 6.40 | 6.40 | 6.40 |
| | | Quaternium-90 Bentonite | Step Lab | | | | | |
| | | (and) Propylene Carbonate | | | | | | |
| C | SuperZinc ® 1000 | Zinc Oxide, Ethyl Ferulate, | Vizor | 15.00 | 0.00 | 15.00 | 0.00 | 0.00 |
| | | Triethoxycaprylyl Silane | | | | | | |
| | SuperZinc Natural | Zinco Oxide, | Vizor | 0.00 | 15.00 | 0.00 | 15.00 | 15.00 |
| | | Polyhydroxystearic Acid | | | | | | |
| | Super Ti 1000 | Titanium Dioxide, Ethyl | Vizor | 2.00 | 0.00 | 0.00 | 2.00 | 0.00 |
| | | Ferulate, Triethoxycapryl | | | | | | |
| | | Silane | | | | | | |
| | Super Ti Natural | Titanium Dioxide, | Vizor | 0.00 | 2.00 | 2.00 | 0.00 | 0.00 |
| | | Polyhydroxystearic Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step Lab | | | | | |
| | NS Boost SiD | Mica, Silica, Dimethicone | Next | 3.00 | 3.00 | 3.00 | 3.00 | 3.00 |
| | | | Step Lab | | | | | |
| | NS Spherilon-10 | Nylon-12 | Next | 1.50 | 1.50 | 1.50 | 1.50 | 1.50 |
| | | | Step Lab | | | | | |
| D | Deionized Water | Water | Any | 19.00 | 19.00 | 19.00 | 19.00 | 19.00 |
| | Magnesium Sulfate | Magnesium Sulfate | Any | 1.00 | 1.00 | 1.00 | 1.00 | 1.00 |
| | NS Phytomoist | Tremella Fuciformis, | Next | 2.00 | 2.00 | 2.00 | 2.00 | 2.00 |
| | | Betain, Glycerin, Water, | Step Lab | | | | | |
| | | Potassium Sorbate, | | | | | | |
| | | Phenoxyethanol | | | | | | |
| | | | | 100.0 | 100.0 | 100.0 | 100.0 | 100.0 |

Note:
this a W/O emulsion

### Lab Procedure

1. Combine phase A ingredients and lightning mix at 500 rpm for 5 minutes.
2. Add phase B to phase A, and lightning mix at 300-500 rpm for 15 minutes to break up agglomerates.
3. Add phase C ingredients to phases AB with homomixing at 3,000-3,500 rpm. Allow to mix for 10-15 minutes. Scrape down the sides of the beaker, and the homogenizer.
4. Combine phase D ingredient in a separate beaker, and lightning mix until uniformly dispersed.
5. Add phase D to phases A-C slowly with homomixing at 3,500 rpm-4,000 rpm; continue mixing for 15 minutes.
6. Drop batch

Set F.

Formula:
Product: Super Thin SPF 50

| Phase | Tradename | Description | Supplier | 1% | 2% | 3% | 4% | 5% |
|---|---|---|---|---|---|---|---|---|
| A | NS Vsol-5 | Isododecane, Hydrogenated | Next | 11.00 | 11.00 | 11.00 | 11.00 | 11.00 |
| | | Polydecene, Dimethicone | Step Lab | | | | | |
| | Jeesile IDD | Isododecane, Dimethicone | Jeen | 6.40 | 6.40 | 6.40 | 6.40 | 6.40 |
| | | Crosspolymer-3 | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) bran | Next | 2.00 | 2.00 | 2.00 | 2.00 | 2.00 |
| | | extract | Step Lab | | | | | |
| | Caprylyl Glycol | Caprylyl Glycol | Any | 0.25 | 0.25 | 0.25 | 0.25 | 0.25 |
| | Phenoxyethanol | Phenoxyethanol | Any | 0.35 | 0.35 | 0.35 | 0.35 | 0.35 |
| B | NS Plant Gel 2002 | Dodecane (and) | Next | 10.00 | 10.00 | 10.00 | 10.00 | 10.00 |
| | | Stearalkonium Bentonite | Step Lab | | | | | |
| | | (and) Triethyl Citrate | | | | | | |
| | Lamiform TGI | Polyglyceryl-3 | Dow | 4.00 | 4.00 | 4.00 | 4.00 | 4.00 |
| | | Diisostearate | Chemical | | | | | |
| | NS Gel IDD | Isododecane(and) | Next | 6.40 | 6.40 | 6.40 | 6.40 | 6.40 |
| | | Quaternium-90 Bentonite | Step Lab | | | | | |
| | | (and) Propylene Carbonate | | | | | | |
| C | SuperZinc ® 1000 | Zinc Oxide, Ethyl Ferulate, | Vizor | 15.00 | 0.00 | 15.00 | 0.00 | 0.00 |
| | | Triethoxycaprylyl Silane | | | | | | |
| | SuperZinc Natural | Zinco Oxide, | Vizor | 0.00 | 15.00 | 0.00 | 15.00 | 15.00 |
| | | Polyhydroxystearic Acid | | | | | | |
| | Super Ti 1000 | Titanium Dioxide, Ethyl | Vizor | 2.00 | 0.00 | 0.00 | 2.00 | 0.00 |
| | | Ferulate, Triethoxycapryl | | | | | | |
| | | Silane | | | | | | |
| | Super Ti Natural | Titanium Dioxide, | Vizor | 0.00 | 2.00 | 2.00 | 0.00 | 0.00 |
| | | Polyhydroxystearic Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step Lab | | | | | |
| | NS Boost SiD | Mica, Silica, Dimethicone | Next | 3.00 | 3.00 | 3.00 | 3.00 | 3.00 |
| | | | Step Lab | | | | | |
| | NS Spherilon-10 | Nylon-12 | Next | 1.50 | 1.50 | 1.50 | 1.50 | 1.50 |
| | | | Step Lab | | | | | |
| D | Deionized Water | Water | Any | 22.00 | 22.00 | 22.00 | 22.00 | 22.00 |
| | Magnesium Sulfate | Magnesium Sulfate | Any | 1.00 | 1.00 | 1.00 | 1.00 | 1.00 |
| | | | | 100.00 | 100.00 | 100.00 | 100.00 | 100.00 |

Note:
this a W/O emulsion

### Lab Procedure

1. Combine phase A ingredients ad lightning mix at 500 rpm for 5 minutes.
2. Add phase B to phase A, and lightning mix at 300-500 rpm for 15 minutes to break up agglomerates
3. Add phase C ingredients to phases AB with homomixing at 3,000-3,500 rpm. Allow to mix for 10-15 minutes. Scrape down the sides of the beaker, and the homogenizer
4. Combine phase D ingredient in a separate beaker, and lightning mix until uniformly dispersed.
5. Add phase D to phases A-C slowly with homomixing at 3,500 rpm-4,000 rpm; continue mixing for 15 minutes.
6. Drop batch

Set G.

Formula:
Product: Super Thin SPF 50

| Phase | Tradename | Description | Supplier | 1% | 2% | 3% | 4% | 5% |
|---|---|---|---|---|---|---|---|---|
| A | NS Vsol-5 | Isododecane, Hydrogenated | Next | 11.00 | 11.00 | 11.00 | 11.00 | 11.00 |
| | | Polydecene, Dimethicone | Step Lab | | | | | |
| | Jeesile IDD | Isododecane, Dimethicone | Jeen | 6.40 | 6.40 | 6.40 | 6.40 | 6.40 |
| | | Crosspolymer-3 | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) bran | Next | 2.00 | 2.00 | 2.00 | 2.00 | 2.00 |
| | | extract | Step Lab | | | | | |
| | Caprylyl Glycol | Caprylyl Glycol | Any | 0.25 | 0.25 | 0.25 | 0.25 | 0.25 |
| | Phenoxyethanol | Phenoxyethanol | Any | 0.35 | 0.35 | 0.35 | 0.35 | 0.35 |
| B | NS Plant Gel 2002 | Dodecane (and) | Next | 10.00 | 10.00 | 10.00 | 10.00 | 10.00 |
| | | Stearalkonium Bentonite | Step Lab | | | | | |
| | | (and) Triethyl Citrate | | | | | | |
| | Lamiform TGI | Polyglyceryl-3 | Dow | 4.00 | 4.00 | 4.00 | 4.00 | 4.00 |
| | | Diisostearate | Chemical | | | | | |
| | NS Gel IDD | Isododecane(and) | Next | 6.40 | 6.40 | 6.40 | 6.40 | 6.40 |
| | | Quaternium-90 Bentonite | Step Lab | | | | | |
| | | (and) Propylene Carbonate | | | | | | |
| C | SuperZinc ® 1000 | Zinc Oxide, Ethyl Ferulate, | Vizor | 15.00 | 0.00 | 15.00 | 0.00 | 0.00 |
| | | Triethoxycaprylyl Silane | | | | | | |
| | SuperZinc Natural | Zinco Oxide, | Vizor | 0.00 | 15.00 | 0.00 | 15.00 | 15.00 |
| | | Polyhydroxystearic Acid | | | | | | |
| | Super Ti 1000 | Titanium Dioxide, Ethyl | Vizor | 2.00 | 0.00 | 0.00 | 2.00 | 0.00 |
| | | Ferulate, Triethoxycapryl | | | | | | |
| | | Silane | | | | | | |
| | Super Ti Natural | Titanium Dioxide, | Vizor | 0.00 | 2.00 | 2.00 | 0.00 | 0.00 |
| | | Polyhydroxystearic Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step Lab | | | | | |
| | NS Boost SiD | Mica, Silica, Dimethicone | Next | 3.00 | 3.00 | 3.00 | 3.00 | 3.00 |
| | | | Step Lab | | | | | |
| | NS Spherilon-10 | Nylon-12 | Next | 1.50 | 1.50 | 1.50 | 1.50 | 1.50 |
| | | | Step Lab | | | | | |
| D | Deionized Water | Water | Any | 22.00 | 22.00 | 22.00 | 22.00 | 22.00 |
| | Magnesium Sulfate | Magnesium Sulfate | Any | 1.00 | 1.00 | 1.00 | 1.00 | 1.00 |
| | | | | 100.00 | 100.00 | 100.00 | 100.00 | 100.00 |

Note:
this a W/O emulsion

1. Combine phase A ingredients and lightning mix at 500 rpm for 5 minutes.
2. Add phase B to phase A, and lightning mix at 300-500 rpm for 15 minutes to break up agglomerates.
3. Add phase C ingredients to phases AB with homomixing at 3,000-3,500 rpm. Allow to mix for 10-15 minutes. Scrape down the sides of the beaker, and the homogenizer
4. Combine phase D ingredient in a separate beaker, and lightning mix until uniformly dispersed.
5. Add phase D to phases A-C slowly with homomixing at 3,500 rpm-4,000 rpm continue mixing for 15 minutes.
6. Drop batch

Set H.

| Formula: | | | | | | | | |
|---|---|---|---|---|---|---|---|---|
| Product: Super Thin SPF 50 Foundation | | | | | | | | |
| Note: this a W/O emulsion | | | | 1 | 2 | 3 | 4 | 5 |
| Phase | Tradename | Description | Supplier | % | % | % | % | % |
| A | NS Vsol-5 | Isododecane, | Next | 5.00 | 5.00 | 5.00 | 5.00 | 5.00 |
| | | Hydrogenated | Step | | | | | |
| | | Polydecene, | Lab | | | | | |
| | | Dimethicone | | | | | | |
| | Jeesile IDD | Isododecane, | Jeen | 6.40 | 6.40 | 6.40 | 6.40 | 6.40 |
| | | Dimethicone | | | | | | |
| | | Crosspolymer-3 | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) | Next | 2.00 | 2.00 | 2.00 | 2.00 | 2.00 |
| | | bran extract | Step | | | | | |
| | | | Lab | | | | | |
| | Caprylyl Glycol | Caprylyl Glycol | Any | 0.25 | 0.25 | 0.25 | 0.25 | 0.25 |
| | Phenoxyethanol | Phenoxyethanol | Any | 0.35 | 0.35 | 0.35 | 0.35 | 0.35 |
| B | NS Plant Gel | Dodecane (and) | Next | 10.00 | 10.00 | 10.00 | 10.00 | 10.00 |
| | 2002 | Stearalkonium | Step | | | | | |
| | | Bentonite (and) | Lab | | | | | |
| | | Triethyl Citrate | | | | | | |
| | Lamiform TGI | Polyglyceryl-3 | Dow | 4.00 | 4.00 | 4.00 | 4.00 | 4.00 |
| | | Diisostearate | Chemical | | | | | |
| | NS Gel IDD | Isododecane(and) | Next | 6.40 | 6.40 | 6.40 | 6.40 | 6.40 |
| | | Quaternium-90 | Step | | | | | |
| | | Bentonite (and) | Lab | | | | | |
| | | Propylene Carbonate | | | | | | |
| C | SuperZinc ® | Zinc Oxide, Ethyl | Vizor | 15.00 | 0.00 | 15.00 | 0.00 | 0.00 |
| | 1000 | Ferulate, | | | | | | |
| | | Triethoxycaprylyl | | | | | | |
| | | Silane | | | | | | |
| | SuperZinc | Zinco Oxide, | Vizor | 0.00 | 15.00 | 0.00 | 15.00 | 15.00 |
| | Natural | Polyhydroxystearic | | | | | | |
| | | Acid | | | | | | |
| | Super Ti 1000 | Titanium Dioxide, | Vizor | 2.00 | 0.00 | 0.00 | 2.00 | 0.00 |
| | | Ethyl Ferulate, | | | | | | |
| | | Triethoxycapryl Silane | | | | | | |
| | Super Ti Natural | Titanium Dioxide, | Vizor | 0.00 | 2.00 | 2.00 | 0.00 | 0.00 |
| | | Polyhydroxystearic | | | | | | |
| | | Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step | | | | | |
| | | | Lab | | | | | |
| | NS Boost SiD | Mica, Silica, | Next | 3.00 | 3.00 | 3.00 | 3.00 | 3.00 |
| | | Dimethicone | Step | | | | | |
| | | | Lab | | | | | |
| | NS Spherilon-10 | Nylon-12 | Next | 1.50 | 1.50 | 1.50 | 1.50 | 1.50 |
| | | | Step | | | | | |
| | | | Lab | | | | | |
| | | Iron oxides | Any | 6.00 | 6.00 | 6.00 | 6.00 | 6.00 |
| D | Deionized Water | Water | Any | 22.00 | 22.00 | 22.00 | 22.00 | 22.00 |
| | Magnesium | Magnesium Sulfate | Any | 1.00 | 1.00 | 1.00 | 1.00 | 1.00 |
| | Sulfate | | | | | | | |
| | | | | 100.00 | 100.0 | 100.0 | 100.0 | 100.0 |

### Lab Procedure

1. Combine phase A ingredients and lightning mix at 500 rpm for 5 minutes.
2. Add phase B to phase A, and lightning mix at 300-500 rpm for 15 minutes to break up agglomerates.
3. Add phase C ingredients to phases AB with homomixing at 3,000-3,500 rpm. Allow to mix for 10-15 minutes. Scrape down the sides of the beaker, and the homogenizer.
4. Combine phase D ingredient in a separate beaker, and lightning mix until uniformly dispersed.
5. Add phase D to phases A-C slowly with homomixing at 3,500 rpm-4,000 rpm continue mixing for 15 minutes.
6. Drop batch.

Set I.

| Formula: | | | | | | | | |
|---|---|---|---|---|---|---|---|---|
| Product: | | | | | | | | |
| Super Thin SPF 50 Foundation | | | | | | | | |
| Note: this a W/O emulsion | | | | 1 | 2 | 3 | 4 | 5 |
| Phase | Tradename | Description | Supplier | % | % | % | % | % |
| A | NS Vsol-5 | Isododecane, | Next | 5.00 | 5.00 | 5.00 | 5.00 | 5.00 |
| | | Hydrogenated | Step Lab | | | | | |
| | | Polydecene, | | | | | | |
| | | Dimethicone | | | | | | |
| | Jeesile IDD | Isododecane, | Jeen | 6.40 | 6.40 | 6.40 | 6.40 | 6.40 |
| | | Dimethicone | | | | | | |
| | | Crosspolymer-3 | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) | Next | 2.00 | 2.00 | 2.00 | 2.00 | 2.00 |
| | | bran extract | Step Lab | | | | | |
| | Caprylyl | Caprylyl Glycol | Any | 0.25 | 0.25 | 0.25 | 0.25 | 0.25 |
| | Glycol | | | | | | | |
| | Phenoxyethanol | Phenoxyethanol | Any | 0.35 | 0.35 | 0.35 | 0.35 | 0.35 |
| B | NS Plant Gel | Dodecane (and) | Next | 10.00 | 10.00 | 10.00 | 10.00 | 10.00 |
| | 2002 | Stearalkonium | Step Lab | | | | | |
| | | Bentonite (and) | | | | | | |
| | | Triethyl Citrate | | | | | | |
| | Lamiform TGI | Polyglyceryl-3 | Dow | 4.00 | 4.00 | 4.00 | 4.00 | 4.00 |
| | | Diisostearate | Chemical | | | | | |
| | NS Gel IDD | Isododecane(and) | Next | 6.40 | 6.40 | 6.40 | 6.40 | 6.40 |
| | | Quaternium-90 | Step Lab | | | | | |
| | | Bentonite (and) | | | | | | |
| | | Propylene | | | | | | |
| | | Carbonate | | | | | | |
| C | SuperZinc ® | Zinc Oxide, Ethyl | Vizor | 15.00 | 0.00 | 15.00 | 0.00 | 0.00 |
| | 1000 | Ferulate, | | | | | | |
| | | Triethoxycaprylyl | | | | | | |
| | | Silane | | | | | | |
| | SuperZinc | Zinco Oxide, | Vizor | 0.00 | 15.00 | 0.00 | 15.00 | 15.00 |
| | Natural | Polyhydroxystearic | | | | | | |
| | | Acid | | | | | | |
| | Super Ti 1000 | Titanium Dioxide, | Vizor | 2.00 | 0.00 | 0.00 | 2.00 | 0.00 |
| | | Ethyl Ferulate, | | | | | | |
| | | Triethoxycapryl | | | | | | |
| | | Silane | | | | | | |
| | Super Ti | Titanium Dioxide, | Vizor | 0.00 | 2.00 | 2.00 | 0.00 | 0.00 |
| | Natural | Polyhydroxystearic | | | | | | |
| | | Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step Lab | | | | | |
| | NS Boost SiD | Mica, Silica, | Next | 3.00 | 3.00 | 3.00 | 3.00 | 3.00 |
| | | Dimethicone | Step Lab | | | | | |
| | NS Spherilon- | Nylon-12 | Next | 1.50 | 1.50 | 1.50 | 1.50 | 1.50 |
| | 10 | | Step Lab | | | | | |
| | Unipure | Iron oxides (red, | Sensient | 6.00 | 6.00 | 6.00 | 6.00 | 6.00 |
| | | yellow, black) | | | | | | |
| D | Deionized | Water | Any | 19.00 | 19.00 | 19.00 | 19.00 | 19.00 |
| | Water | | | | | | | |
| | Magnesium | Magnesium | Any | 1.00 | 1.00 | 1.00 | 1.00 | 1.00 |
| | Sulfate | Sulfate | | | | | | |
| | NS Phytomoist | Tremella | Next | 3.00 | 3.00 | 3.00 | 3.00 | 3.00 |
| | | Fuciformis, Betain, | Step Lab | | | | | |
| | | Glycerin, Water, | | | | | | |
| | | Potassium Sorbate, | | | | | | |
| | | Phenoxyethanol | | | | | | |
| | | | | 100.00 | 100.00 | 100.00 | 100.00 | 100.00 |

### Lab Procedure

1. Combine phase A ingredients and lightning mix at 500 rpm for 5 minutes.
2. Add phase B to phase A, and lightning mix at 300-500 rpm for 15 minutes to break−up agglomerates
3. Add phase C ingredients to phases AB with homomixing at 3,000-3,500 rpm. Allow to mix for 10-15 minutes. Scrape down the sides of the beaker, and the homogenizer
4. Combine phase D ingredient in a separate beaker, and lightning mix until uniformly dispersed.
5. Add phase D to phases A-C slowly with homomixing at 3,500 rpm-4,000 rpm; continue mixing for 15 minutes
6. Drop batch

Set J.

| Formula: | | | | | | | | |
|---|---|---|---|---|---|---|---|---|
| Product: | | | | | | | | |
| SPF 50 Cream Foundation | | | | | | | | |
| Note: this a W/O emulsion | | | | 1 | 2 | 3 | 4 | 5 |
| Phase | Tradename | Description | Supplier | % | % | % | % | % |
| A | NS Vsol-5 | Isododecane, | Next | 3.00 | 3.00 | 3.00 | 3.00 | 4.00 |
| | | Hydrogenated | Step Lab | | | | | |
| | | Polydecene, | | | | | | |
| | | Dimethicone | | | | | | |
| | Jeesile IDD | Isododecane, | Jeen | 6.40 | 6.40 | 6.40 | 6.40 | 6.40 |
| | | Dimethicone | | | | | | |
| | | Crosspolymer-3 | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) | Next | 2.00 | 2.00 | 2.00 | 2.00 | 2.00 |
| | | bran extract | Step Lab | | | | | |
| | Caprylyl | Caprylyl Glycol | Any | 0.25 | 0.25 | 0.25 | 0.25 | 0.25 |
| | Glycol | | | | | | | |
| | Phenoxyethanol | Phenoxyethanol | Any | 0.35 | 0.35 | 0.35 | 0.35 | 0.35 |
| B | NS Plant Gel | Dodecane (and) | Next | 10.00 | 10.00 | 10.00 | 10.00 | 10.00 |
| | 2002 | Stearalkonium | Step Lab | | | | | |
| | | Bentonite (and) | | | | | | |
| | | Triethyl Citrate | | | | | | |
| | Span 80 | Sorbitan Oleate | Croda | 4.00 | 2.00 | 4.00 | 3.00 | 1.00 |
| | Sisterna SP30- | Sucrose Distearate | Sisterna | 2.00 | 4.00 | 2.00 | 3.00 | 4.00 |
| | C | | | | | | | |
| | NS Gel IDD | Isododecane(and) | Next | 6.40 | 6.40 | 6.40 | 6.40 | 6.40 |
| | | Quaternium-90 | Step Lab | | | | | |
| | | Bentonite (and) | | | | | | |
| | | Propylene | | | | | | |
| | | Carbonate | | | | | | |
| C | SuperZinc ® | Zinc Oxide, Ethyl | Vizor | 15.00 | 0.00 | 15.00 | 0.00 | 0.00 |
| | 1000 | Ferulate, | | | | | | |
| | | Triethoxycaprylyl | | | | | | |
| | | Silane | | | | | | |
| | SuperZinc | Zinco Oxide, | Vizor | 0.00 | 15.00 | 0.00 | 15.00 | 15.00 |
| | Natural | Polyhydroxystearic | | | | | | |
| | | Acid | | | | | | |
| | Super Ti 1000 | Titanium Dioxide, | Vizor | 2.00 | 0.00 | 0.00 | 2.00 | 0.00 |
| | | Ethyl Ferulate, | | | | | | |
| | | Triethoxycapryl | | | | | | |
| | | Silane | | | | | | |
| | Super Ti | Titanium Dioxide, | Vizor | 0.00 | 2.00 | 2.00 | 0.00 | 0.00 |
| | Natural | Polyhydroxystearic | | | | | | |
| | | Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step Lab | | | | | |
| | NS Boost SiD | Mica, Silica, | Next | 3.00 | 3.00 | 3.00 | 3.00 | 3.00 |
| | | Dimethicone | Step Lab | | | | | |
| | NS Spherilon- | Nylon-12 | Next | 1.50 | 1.50 | 1.50 | 1.50 | 1.50 |
| | 10 | | Step Lab | | | | | |
| | Unipure | Iron oxides red, | Sensient | 6.00 | 6.00 | 6.00 | 6.00 | 6.00 |
| | | yellow, black) | | | | | | |
| D | Deionized | Water | Any | 19.00 | 19.00 | 19.00 | 19.00 | 19.00 |
| | Water | | | | | | | |
| | Magnesium | Magnesium | Any | 1.00 | 1.00 | 1.00 | 1.00 | 1.00 |
| | Sulfate | Sulfate | | | | | | |
| | NS Phytomoist | Tremella | Next | 3.00 | 3.00 | 3.00 | 3.00 | 3.00 |
| | | Fuciformis, Betain, | Step Lab | | | | | |
| | | Glycerin, Water, | | | | | | |
| | | Potassium Sorbate, | | | | | | |
| | | Phenoxyethanol | | | | | | |
| | | | | 100.00 | 100.00 | 100.00 | 100.00 | 100.00 |

### Lab Procedure

1. Combine phase A ingredients and lightning mix at 500 rpm for 5 minutes.
2. Add phase B to phase A, and lightning mix at 300-500 rpm for 15 minutes to break up agglomerates.
3. Add phase C ingredients to phases AB with homomixing at 3,000-3,500 rpm. Allow to mix for 10-15 minutes. Scrape down the sides of the beaker, and the homogenizer.
4. Combine phase D ingredient in a separate beaker, and lightning mix until uniformly dispersed.
5. Add phase D to phases A-C slowly with homomixing at 3,500 rpm-4,000 rpm; continue mixing for 15 minutes.
6. Drop batch

Set K.

| Formula: | | | | | | | | |
|---|---|---|---|---|---|---|---|---|
| Product: | | | | | | | | |
| Super Thin SPF 50 | | | | | | | | |
| Note: this a W/O emulsion | | | | 1 | 2 | 3 | 4 | 5 |
| Phase | Tradename | Description | Supplier | % | % | % | % | % |
| A | NS Vsol-5 | Isododecane, | Next | 7.00 | 7.00 | 7.00 | 7.00 | 7.00 |
| | | Hydrogenated | Step Lab | | | | | |
| | | Polydecene, | | | | | | |
| | | Dimethicone | | | | | | |
| | Jeesile IDD | Isododecane, | Jeen | 6.40 | 6.40 | 6.40 | 6.40 | 6.40 |
| | | Dimethicone | | | | | | |
| | | Crosspolymer-3 | | | | | | |
| | 5 CST | Dimethicone | Any | 8.00 | 8.00 | 8.00 | 8.00 | 8.00 |
| | Dermol TDSA | Tridecyl Salicylate | ALZO | 5.80 | 5.80 | 5.30 | 5.30 | 5.30 |
| | SolarYze | Oryza sativa (rice) | Next | 2.00 | 2.00 | 2.00 | 2.00 | 2.00 |
| | | bran extract | Step Lab | | | | | |
| | Caprylyl | Caprylyl Glycol | Any | 0.25 | 0.25 | 0.25 | 0.25 | 0.25 |
| | Glycol | | | | | | | |
| | Phenoxyethanol | Phenoxyethanol | Any | 0.35 | 0.35 | 0.35 | 0.35 | 0.35 |
| B | NS Plant Gel | Dodecane (and) | Next | 10.00 | 10.00 | 10.00 | 10.00 | 10.00 |
| | 2002 | Stearalkonium | Step Lab | | | | | |
| | | Bentonite (and) | | | | | | |
| | | Triethyl Citrate | | | | | | |
| | Abil EM 90 | Cetyl PEG/PPG- | Evonik | 3.00 | 3.00 | 3.00 | 3.00 | 3.00 |
| | | 10/1 Dimethicone | | | | | | |
| | NS Gel IDD | Isododecane(and) | Next | 6.40 | 6.40 | 6.40 | 6.40 | 6.40 |
| | | Quaternium-90 | Step Lab | | | | | |
| | | Bentonite (and) | | | | | | |
| | | Propylene | | | | | | |
| | | Carbonate | | | | | | |
| | Neo Heliopan | Isoamyl p- | Symrise | 1.00 | 1.00 | 1.00 | 1.00 | 1.00 |
| | MBC | Methoxycinnamate | | | | | | |
| | Neo Heliopan | Avobenzone | Symrise | 1.00 | 1.00 | 1.00 | 1.00 | 1.00 |
| | 357 | | | | | | | |
| | Neo Heliopan | Octocrylene | Symrise | 2.00 | 2.00 | 2.00 | 2.00 | 2.00 |
| | 303 | | | | | | | |
| | Neo Heliopan | Homosalate | Symrise | 2.00 | 2.00 | 2.00 | 2.00 | 2.00 |
| | OS | | | | | | | |
| | Neo Heliopan | Octinoxate | Symrise | 2.00 | 2.00 | 2.00 | 2.00 | 2.00 |
| | AV | | | | | | | |
| | Neo Heliopan | Octisalate | Symrise | 2.00 | 2.00 | 2.00 | 2.00 | 2.00 |
| | OS | | | | | | | |
| C | SuperZinc ® | Zinc Oxide, Ethyl | Vizor | 13.00 | 13.00 | 13.00 | 13.00 | 13.00 |
| | 1000 | Ferulate, | | | | | | |
| | | Triethoxycaprylyl | | | | | | |
| | | Silane | | | | | | |
| | SuperZinc | Zinco Oxide, | Vizor | 0.00 | 1.00 | 0.00 | 0.00 | 0.00 |
| | Natural | Polyhydroxystearic | | | | | | |
| | | Acid | | | | | | |
| | Super Ti 1000 | Titanium Dioxide, | Vizor | 0.00 | 0.00 | 2.00 | 0.00 | 0.00 |
| | | Ethyl Ferulate, | | | | | | |
| | | Triethoxycapryl | | | | | | |
| | | Silane | | | | | | |
| | Super Ti | Titanium Dioxide, | Vizor | 0.00 | 0.00 | 0.00 | 2.00 | 0.00 |
| | Natural | Polyhydroxystearic | | | | | | |
| | | Acid | | | | | | |
| | MT-500 | Titanium Dioxide | Presperse | 0.00 | 0.00 | 0.00 | 0.00 | 2.00 |
| | NS PGPR | Polyglyceryl 3 | Next | 0.30 | 0.30 | 0.30 | 0.30 | 0.30 |
| | | Polyricinoleate | Step Lab | | | | | |
| | NS Boost SiD | Mica, Silica, | Next | 3.00 | 3.00 | 3.00 | 3.00 | 3.00 |
| | | Dimethicone | Step Lab | | | | | |
| | NS Spherilon- | Nylon-12 | Next | 1.50 | 1.50 | 1.50 | 1.50 | 1.50 |
| | 10 | | Step Lab | | | | | |
| D | Deionized | Water | Any | 22.00 | 21.00 | 20.50 | 20.50 | 20.50 |
| | Water | | | | | | | |
| | Magnesium | Magnesium | Any | 1.00 | 1.00 | 1.00 | 1.00 | 1.00 |
| | Sulfate | Sulfate | | | | | | |
| | | | | 100.00 | 100.00 | 100.00 | 100.00 | 100.00 |

### Lab Procedure

1. Combine phase A ingredients and lightning mix at 500 rpm for 5 minutes.
2. Add phase B to phase A, and lightning mix at 300-500 rpm for 15 minutes to break up agglomerates
3. Add phase C ingredients to phases AB with homomixing at 3,000-3,500 rpm. Allow to mix for 10-15 minutes. Scrape down the sides of the beaker, and the homogenizer
4. Combine phase D ingredient in a separate beaker, and lightning mix until uniformly dispersed.
5. Add phase D to phases A-C slowly with homomixing at 3,500 rpm-4,000 rpm, continue mixing for 15 minutes
6. Drop batch

Set L.

| 0_W Emulsion | | | | | | | | |
|---|---|---|---|---|---|---|---|---|
| Phase | | | | | | | | |
| A | Ingredients | INCI Name | Supplier | 1 | 2 | 3 | 4 | 5 |
| A | Water | Water | Any | 56.0 | 56.0 | 56.0 | 56.0 | 56.0 |
| B | Veegum Ultra | Magnesium Aluminium | Veegum ® Ultra | 2.0 | 2.0 | 2.0 | 2.0 | 2.0 |
| | Granules | Silicate | | | | | | |
| | Glycerin | Glycerine | Any | 4.0 | 4.0 | 4.0 | 4.0 | 4.0 |
| | Zemea | Propanediol | Dupont | 3.0 | 3.0 | 3.0 | 3.0 | 3.0 |
| | Propanediol | | | | | | | |
| C | Desert Date | Balanites Roxburghii | EPHYLA/NSL | 2.0 | 2.0 | 2.0 | 2.0 | 2.0 |
| | | Seed Oil | | | | | | |
| | HTRE | Helianthus Annuus Seed | Ephyla/nsl | 2.0 | 2.0 | 2.0 | 2.0 | 2.0 |
| | Dermol 816 | Ethyhexyl Palmitate | Croda | 5.0 | 5.0 | 5.0 | 5.0 | 5.0 |
| | Arlacel 165 | PEG-100 Stearate, GMS | Croda | 3.0 | 3.0 | 3.0 | 3.0 | 3.0 |
| | NS EX-81 | Polyglyceryl-8 Oleate | Next Step Lab | 1.0 | 1.0 | 1.0 | 1.0 | 1.0 |
| | E335 | PVP/VA Copolymer | Ashland | 2.0 | 2.0 | 2.0 | 2.0 | 2.0 |
| | Lanette 22 | Behenyl Alcohol | BASF | 2.0 | 2.0 | 2.0 | 2.0 | 2.0 |
| D | SuperZinc ® | Zinc Oxide, Ethyl | Vizor | 15.0 | 0.0 | 15.0 | 0.0 | 0.0 |
| | 1000 | Ferulate, | | | | | | |
| | | Triethoxycaprylyl Silane | | | | | | |
| | SuperZinc | Zinco Oxide, | Vizor | 0.0 | 15.0 | 0.0 | 15.0 | 15.0 |
| | Natural | Polyhydroxystearic Acid | | | | | | |
| | Super Ti 1000 | Titanium Dioxide, Ethyl | Vizor | 2.0 | 0.0 | 0.0 | 2.0 | 0.0 |
| | | Ferulate, Triethoxycapryl | | | | | | |
| | | Silane | | | | | | |
| | Super Ti Natural | Titanium Dioxide, | Vizor | 0.0 | 2.0 | 2.0 | 0.0 | 0.0 |
| | | Polyhydroxystearic Acid | | | | | | |
| | MT-500 | Titanium Dioxide | Presperse | 0.0 | 0.0 | 0.0 | 0.0 | 2.0 |
| E | Phenoxyethanol | Phenoxyethanol | Any | 1.0 | 1.0 | 1.0 | 1.0 | 1.0 |
| | | | | 100 | 100 | 100 | 100 | 100 |

### Procedure:

1. Combine Phase A and B and homomix until gum is completely dispersed
2. Combine phase C and heat to 75-80 C.
3. Add Phase D to phase C and homomix for 30-40 mins until zinc oxide is completely dispersed.
4. Heat Phase C and D to 75-80 C and add Phase E while homoxing.
5. Add Phase C and D to Phase AB slowly at 75-80 C with homogenizing.
6. Homomix and air cool to 60 C then add phase E.
7. Transfer batch to a sweep mixer at 55 C and sweep mix while air cooling to 35 C.

Set M.

| O-W Emulsion | | | | | | | | |
|---|---|---|---|---|---|---|---|---|
| Phase | | | | | | | | |
| A | Ingredients | INCI Name | Supplier | 1 | 2 | 3 | 4 | 5 |
| A | Water | Water | Any | 53.0 | 53.0 | 53.0 | 53.0 | 53.0 |
| B | Veegum Ultra | Magnesium Aluminium | Veegum ® | 2.0 | 2.0 | 2.0 | 2.0 | 2.0 |
| | Granules | Silicate | Ultra | | | | | |
| | Glycerin | Glycerine | Any | 6.0 | 6.0 | 6.0 | 6.0 | 6.0 |
| | Zemea | Propanediol | Dupont | 3.0 | 3.0 | 3.0 | 3.0 | 3.0 |
| | Propanediol | | | | | | | |
| C | Desert Date | Balanites Roxburghii | EPHYLA/NSL | 2.0 | 2.0 | 2.0 | 2.0 | 2.0 |
| | | Seed Oil | | | | | | |
| | HTRE | Helianthus Annuus | Ephyla/nsl | 2.0 | 2.0 | 2.0 | 2.0 | 2.0 |
| | | Seed | | | | | | |
| | Dermol 816 | Ethyhexyl Palmitate | Croda | 5.0 | 5.0 | 5.0 | 5.0 | 5.0 |
| | Arlacel 165 | PEG-100 Stearate, | Croda | 3.0 | 3.0 | 3.0 | 3.0 | 3.0 |
| | | GMS | | | | | | |
| | NS EX-81 | Polyglyceryl-8 Oleate | Next Step Lab | 1.0 | 1.0 | 1.0 | 1.0 | 1.0 |
| | Lanette 22 | Behenyl Alcohol | BASF | 2.0 | 2.0 | 2.0 | 2.0 | 2.0 |
| D | SuperZinc ® | Zinc Oxide, Ethyl | Vizor | 15.0 | 0.0 | 15.0 | 0.0 | 0.0 |
| | 1000 | Ferulate, | | | | | | |
| | | Triethoxycaprylyl | | | | | | |
| | | Silane | | | | | | |
| | SuperZinc | Zinco Oxide, | Vizor | 0.0 | 15.0 | 0.0 | 15.0 | 15.0 |
| | Natural | Polyhydroxystearic | | | | | | |
| | | Acid | | | | | | |
| | Super Ti 1000 | Titanium Dioxide, | Vizor | 2.0 | 0.0 | 0.0 | 2.0 | 0.0 |
| | | Ethyl Ferulate, | | | | | | |
| | | Triethoxycapryl Silane | | | | | | |
| | Super Ti | Titanium Dioxide, | Vizor | 0.0 | 2.0 | 2.0 | 0.0 | 0.0 |
| | Natural | Polyhydroxystearic | | | | | | |
| | | Acid | | | | | | |
| | MT-500 | Titanium Dioxide | Presperse | 0.0 | 0.0 | 0.0 | 0.0 | 2.0 |
| E | Phenoxyethanol | Phenoxyethanol | Any | 1.0 | 1.0 | 1.0 | 1.0 | 1.0 |
| F | Ultrapure | Polyvinyl Acetate | HB Fuller | 3.0 | 3.0 | 3.0 | 3.0 | 3.0 |
| | | Homopolymer | | | | | | |
| | | | | 100.0 | 100 | 100 | 100 | 100 |

### Procedure:

1. Combine Phase A and B and homomix until gum is completely dispersed.
2. Combine phase C and heat to 75-80 C.
3. Add Phase D to phase C and homomix for 30-40 mins until zinc oxide is completely dispersed.
4. Heat Phase C and D to 75-80 C and add Phase E while homoxing.
5. Add Phase C and D to Phase AB slowly at 75-80 C with homogenizing.
6. Homomix and air cool to 60 C then add phase E.
7. Transfer batch to a sweep mixer at 55 C and sweep mix while air cooling to 35 C.

Set N.

| O-W Emulsion SPF-50 | | | | | | | | |
|---|---|---|---|---|---|---|---|---|
| Phase | | | | | | | | |
| A | Ingredients | INCI Name | Supplier | 1 | 2 | 3 | 4 | 5 |
| A | Water | Water | Any | 51.0 | 51.0 | 51.0 | 51.0 | 51.0 |
| B | Veegum Ultra | Magnesium | Veegum ® | 2.0 | 2.0 | 2.0 | 2.0 | 2.0 |
| | Granules | Aluminium Silicate | Ultra | | | | | |
| | Glycerin | Glycerine | Any | 6.0 | 6.0 | 6.0 | 6.0 | 6.0 |
| | Zemea | Propanediol | Dupont | 3.0 | 3.0 | 3.0 | 3.0 | 3.0 |
| | Propanediol | | | | | | | |
| C | Desert Date | Balanites Roxburghii | EPHYLA/NSL | 2.0 | 2.0 | 2.0 | 2.0 | 2.0 |
| | | Seed Oil | | | | | | |
| | HTRE | Helianthus Annuus | Ephyla/nsl | 2.0 | 2.0 | 2.0 | 2.0 | 2.0 |
| | | Seed | | | | | | |
| | Dermol 816 | Ethyhexyl Palmitate | Croda | 5.0 | 5.0 | 5.0 | 5.0 | 5.0 |
| | Brij 721 | Steareth-21 | Croda | 4.0 | 4.0 | 4.0 | 4.0 | 4.0 |
| | Brij 21 | Steareth-2 | Croda | 2.0 | 2.0 | 2.0 | 2.0 | 2.0 |
| | Lanette 22 | Behenyl Alcohol | BASF | 2.0 | 2.0 | 2.0 | 2.0 | 2.0 |
| C | SuperZinc ® | Zinc Oxide, Ethyl | Vizor | 17.0 | 15.0 | 0.0 | 10.0 | 2.0 |
| | 1000-P | Ferulate, | | | | | | |
| | | Phenyltriethoxy | | | | | | |
| | | Silane | | | | | | |
| | Super Ti 1000- | Titanium Dioxide, | Vizor | 0.0 | 2.0 | 17.0 | 10.0 | 17.0 |
| | P2 | Ethyl Ferulate, | | | | | | |
| | | Diphenyldiethoxy | | | | | | |
| | | Silane | | | | | | |
| | MT-500 | Titanium Dioxide | Presperse | 0.0 | 0.0 | 0.0 | 0.0 | 2.0 |
| E | Phenoxyethanol | Phenoxyethanol | Any | 1.0 | 1.0 | 1.0 | 1.0 | 1.0 |
| F | Ultrapure | Polyvinyl Acetate | HB Fuller | 3.0 | 3.0 | 3.0 | 3.0 | 3.0 |
| | | Homopolymer | | | | | | |
| | | | | 100.0 | 100 | 100 | 100 | 100 |

### Procedure:

1. Combine Phase A and B and homomix until gum is completely dispersed
2. Combine phase C and heat to 75-80 C.
3. Add Phase D to phase C and homomix for 30-40 mins until zinc oxide is completely dispersed.
4. Heat Phase C and D to 75-80 C and add Phase E while homoxing.
5. Add Phase C and D to Phase AB slowly at 75-80° C. with homogenizing.
6. Homomix and air cool to 60 C then add phase E.
7. Transfer batch to a sweep mixer at 55 C and sweep mix while air cooling to 35 C.

Set O.

| O-W Emulsion SPF-50 | | | | | | | | |
|---|---|---|---|---|---|---|---|---|
| Phase | | | | | | | | |
| A | Ingredients | INCI Name | Supplier | 1 | 2 | 3 | 4 | 5 |
| A | Water | Water | Any | 51.0 | 51.0 | 51.0 | 51.0 | 51.0 |
| B | Veegum Ultra | Magnesium Aluminium | Veegum ® | 2.0 | 2.0 | 2.0 | 2.0 | 2.0 |
| | Granules | Silicate | Ultra | | | | | |
| | Glycerin | Glycerine | Any | 6.0 | 6.0 | 6.0 | 6.0 | 6.0 |
| | Zemea | Propanediol | Dupont | 3.0 | 3.0 | 3.0 | 3.0 | 3.0 |
| | Propanediol | | | | | | | |
| C | Desert Date | Balanites Roxburghii | EPHYLA/NSL | 2.0 | 2.0 | 2.0 | 2.0 | 2.0 |
| | | Seed Oil | | | | | | |
| | HTRE | Helianthus Annuus Seed | Ephyla/nsl | 2.0 | 2.0 | 2.0 | 2.0 | 2.0 |
| | Dermol 816 | Ethyhexyl Palmitate | Croda | 5.0 | 5.0 | 5.0 | 5.0 | 5.0 |
| | Brij 721 | Steareth-21 | Croda | 4.0 | 4.0 | 4.0 | 4.0 | 4.0 |
| | Brij 21 | Steareth-2 | Croda | 2.0 | 2.0 | 2.0 | 2.0 | 2.0 |
| | Lanette 22 | Behenyl Alcohol | BASF | 2.0 | 2.0 | 2.0 | 2.0 | 2.0 |
| C | SuperZinc ® | Zinc Oxide, Ethyl | Vizor | 17.0 | 15.0 | 0.0 | 10.0 | 2.0 |
| | 1000-P | Ferulate, Phenyltriethoxy | | | | | | |
| | | Silane | | | | | | |
| | Super Ti 1000- | Titanium Dioxide, Ethyl | Vizor | 0.0 | 2.0 | 17.0 | 10.0 | 17.0 |
| | P2 | Ferulate, | | | | | | |
| | | Diphenyldiethoxy Silane | | | | | | |
| | MT-500 | Titanium Dioxide | Presperse | 0.0 | 0.0 | 0.0 | 0.0 | 2.0 |
| E | Phenoxyethanol | Phenoxyethanol | Any | 1.0 | 1.0 | 1.0 | 1.0 | 1.0 |
| F | Ultrapure | Polyvinyl Acetate | HB Fuller | 3.0 | 3.0 | 3.0 | 3.0 | 3.0 |
| | | Homopolymer | | | | | | |
| | | | | 100.0 | 100 | 100 | 100 | 100 |

### Procedure:

1. Combine Phase A and B and homomix until gum is completely dispersed.
2. Combine phase C and heat to 75-80 C.
3. Add Phase D to phase C and homomix for 30-40 mins until zinc oxide is completely dispersed.
4. Heat Phase C and D to 75-80 C and add Phase E while homoxing.
5. Add Phase C and D to Phase AB slowly at 75-80 C with homogenizing.
6. Homomix and air cool to 60 C then add phase E.
7. Transfer batch to a sweep mixer at 55 C and sweep mix while air cooling to 35 C.

Set P.

| O-W Emulsion SPF-50 Hybrid | | | | | | | | |
|---|---|---|---|---|---|---|---|---|
| Phase | | | | | | | | |
| A | Ingredients | INCI Name | Supplier | 1 | 2 | 3 | 4 | 5 |
| A | Water | Water | Any | 51.0 | 51.0 | 51.0 | 51.0 | 51.0 |
| B | Veegum Ultra | Magnesium Aluminium | Veegum ® | 2.0 | 2.0 | 2.0 | 2.0 | 2.0 |
| | Granules | Silicate | Ultra | | | | | |
| | Glycerin | Glycerine | Any | 3.0 | 3.0 | 3.0 | 3.0 | 3.0 |
| | Zemea | Propanediol | Dupont | 3.0 | 3.0 | 3.0 | 3.0 | 3.0 |
| | Propanediol | | | | | | | |
| C | Desert Date | Balanites Roxburghii Seed Oil | EPHYLA/NSL | 1.0 | 1.0 | 1.0 | 1.0 | 1.0 |
| | HTRE | Helianthus Annuus Seed | Ephyla/nsl | 1.0 | 1.0 | 1.0 | 1.0 | 1.0 |
| | Neo Heliopan | Octocrylene | Symrise | 2.0 | 2.0 | 2.0 | 2.0 | 2.0 |
| | 303 | | | | | | | |
| | Neo Heliopan | Homosalate | Symrise | 2.0 | 2.0 | 2.0 | 2.0 | 2.0 |
| | OS | | | | | | | |
| | Neo Heliopan | Octinoxate | Symrise | 2.0 | 2.0 | 2.0 | 2.0 | 2.0 |
| | AV | | | | | | | |
| | Neo Heliopan | Octisalate | Symrise | 2.0 | 2.0 | 2.0 | 2.0 | 2.0 |
| | OS | | | | | | | |
| | Dermol 816 | Ethyhexyl Palmitate | Croda | 2.0 | 4.0 | 2.0 | 2.0 | 2.0 |
| | Brij 721 | Steareth-21 | Croda | 4.0 | 4.0 | 4.0 | 4.0 | 4.0 |
| | Brij 21 | Steareth-2 | Croda | 2.0 | 2.0 | 2.0 | 2.0 | 2.0 |
| | Lanette 22 | Behenyl Alcohol | BASF | 2.0 | 2.0 | 2.0 | 2.0 | 2.0 |
| C | SuperZinc ® | Zinc Oxide, Ethyl Ferulate, | Vizor | 17.0 | 13.0 | 0.0 | 10.0 | 2.0 |
| | 1000-P | Phenyltriethoxy Silane | | | | | | |
| | Super Ti 1000- | Titanium Dioxide, Ethyl | Vizor | 0.0 | 2.0 | 17.0 | 10.0 | 17.0 |
| | P2 | Ferulate, Diphenyldiethoxy | | | | | | |
| | | Silane | | | | | | |
| | MT-500 | Titanium Dioxide | Presperse | 0.0 | 0.0 | 0.0 | 0.0 | 2.0 |
| E | Phenoxyethanol | Phenoxyethanol | Any | 1.0 | 1.0 | 1.0 | 1.0 | 1.0 |
| F | Ultrapure | Polyvinyl Acetate | HB Fuller | 3.0 | 3.0 | 3.0 | 3.0 | 3.0 |
| | | Homopolymer | | | | | | |
| | | | | 100.0 | 100 | 100 | 100 | 100 |

### Procedure:

1. Combine Phase A and B and homomix until gum is completely dispersed
2. Combine phase C and heat to 75-80 C
3. Add Phase D to phase C and homomix for 3040 mins until zinc oxide is completely dispersed
4. Heat Phase C and D to 75-80 C and add Phase E while homoxing
5. Add Phase C and D to Phase AB slowly at 75-80 C with homogenizing
6. Homomix and air cool to 60 C then add phase E
7. Transfer batch to a sweep mixer at 55 C and sweep mix while air cooling to 35 C

Set Q.

| Formula: | | | | | | | | |
|---|---|---|---|---|---|---|---|---|
| Product: SPF-50 Stick | | | | 1 | 2 | 3 | 4 | 5 |
| Phase | Tradename | Description | Supplier | % | % | % | % | % |
| A | Dermol IPP | Isopropyl Palmitate | Alzo | 25.00 | 25.00 | 25.00 | 25.00 | 25.00 |
| | Neobee M | Caprylic/Capric | Stepan | 25.00 | 25.00 | 25.00 | 25.00 | 25.00 |
| | | Triglyceride | | | | | | |
| | 5 CST | Dimethicone | Any | 9.50 | 9.50 | 9.50 | 9.50 | 9.50 |
| | Dermol | Tridecyl Salicylate | ALZO | 10.20 | 10.20 | 10.20 | 10.20 | 10.20 |
| | TDSA | | | | | | | |
| | SolarYze | Oryza sativa (rice) | Next Step | 2.00 | 2.00 | 2.00 | 2.00 | 2.00 |
| | | bran extract | Lab | | | | | |
| | Jeenate 3H | Polyethylene | Jeen | 8.00 | 8.00 | 8.00 | 8.00 | 8.00 |
| C | SuperZinc ® | Zinc Oxide, Ethyl | Vizor | 15.00 | 0.00 | 15.00 | 0.00 | 0.00 |
| | 1000 | Ferulate, | | | | | | |
| | | Triethoxycaprylyl | | | | | | |
| | | Silane | | | | | | |
| | SuperZinc | Zinco Oxide, | Vizor | 0.00 | 15.00 | 0.00 | 15.00 | 15.00 |
| | Natural | Polyhydroxystearic | | | | | | |
| | | Acid | | | | | | |
| | Super Ti | Titanium Dioxide, | Vizor | 2.00 | 0.00 | 0.00 | 2.00 | 0.00 |
| | 1000 | Ethyl Ferulate, | | | | | | |
| | | Triethoxycapryl | | | | | | |
| | | Silane | | | | | | |
| | Super Ti | Titanium Dioxide, | Vizor | 0.00 | 2.00 | 2.00 | 0.00 | 0.00 |
| | Natural | Polyhydroxystearic | | | | | | |
| | | Acid | | | | | | |
| | MT-500 | Titanium Dioxide | Presperse | 0.00 | 0.00 | 0.00 | 0.00 | 2.00 |
| | NS PGPR | Polyglyceryl 3 | Next Step | 0.30 | 0.30 | 0.30 | 0.30 | 0.30 |
| | | Polyricinoleate | Lab | | | | | |
| | NS Boost | Mica, Silica, | Next Step | 3.00 | 3.00 | 3.00 | 3.00 | 3.00 |
| | SiD | Dimethicone | Lab | | | | | |
| | | | | 100.00 | 100.00 | 100.00 | 100.00 | 100.00 |

### Lab Procedure

1. Combine phase A ingredients and lightning mix at 500 rpm for 5 minutes.
2. Add phase B to phase A, and lightning mix at 300-500 rpm for 15 minutes to break up agglomerates
3. Add phase C ingredients to phases AB with homomixing at 3,000-3,500 rpm. Allow to mix for 10-15 minutes. Scrape down the sides of the beaker, and the homogenizer
4. Combine phase D ingredient in a separate beaker, and lightning mix until uniformly dispersed.
5. Add phase D to phases A-C slowly with homomixing at 3,500 rpm-4,000 rpm; continue mixing for 15 minutes
6. Drop batch

Set R.

| Formula: | | | | | | | | |
|---|---|---|---|---|---|---|---|---|
| Product: SPF 50 Stick | | | | 1 | 2 | 3 | 4 | 5 |
| Phase | Tradename | Description | Supplier | % | % | % | % | % |
| A | Dermol IPP | Isopropyl Palmitate | Alzo | 11.00 | 11.00 | 11.00 | 11.00 | 11.00 |
| | Neobee M | Caprylic/Capric | Stepan | 16.90 | 16.90 | 16.90 | 16.90 | 16.09 |
| | | Triglyceride | | | | | | |
| | Dermol 816 | Ethyl Hexyl Palmitate | Alzo | 20.00 | 20.00 | 20.00 | 20.00 | 20.00 |
| | 5 CST | Dimethicone | Any | 9.50 | 9.50 | 9.50 | 9.50 | 9.50 |
| | Dermol | Tridecyl Salicylate | ALZO | 5.30 | 5.30 | 5.30 | 5.30 | 5.30 |
| | TDSA | | | | | | | |
| | SolarYze | Oryza sativa (rice) bran | Next Step | 2.00 | 2.00 | 2.00 | 2.00 | 2.00 |
| | | extract | Lab | | | | | |
| B | SP-63 | Carnauba Wax | Strah&Pitsch | 2.00 | 2.00 | 2.00 | 2.00 | 2.00 |
| | Jeenate 3H | Polyethylene | Jeen | 6.00 | 6.00 | 6.00 | 6.00 | 6.00 |
| | NS Gel | Octyldodecanol, | Next Step | 10.00 | 10.00 | 10.00 | 10.00 | 10.00 |
| | EUG | Quaternium-90 | Lab | | | | | |
| | | Bentonite, Propylene | | | | | | |
| | | Carbonate | | | | | | |
| C | SuperZinc ® | Zinc Oxide, Ethyl | Vizor | 15.00 | 0.00 | 15.00 | 0.00 | 0.00 |
| | 1000 | Ferulate, | | | | | | |
| | | Triethoxycaprylyl | | | | | | |
| | | Silane | | | | | | |
| | SuperZinc | Zinco Oxide, | Vizor | 0.00 | 15.00 | 0.00 | 15.00 | 15.00 |
| | Natural | Polyhydroxystearic | | | | | | |
| | | Acid | | | | | | |
| | Super Ti | Titanium Dioxide, Ethyl | Vizor | 2.00 | 0.00 | 0.00 | 2.00 | 0.00 |
| | 1000 | Ferulate, | | | | | | |
| | | Triethoxycapryl Silane | | | | | | |
| | Super Ti | Titanium Dioxide, | Vizor | 0.00 | 2.00 | 2.00 | 0.00 | 0.00 |
| | Natural | Polyhydroxystearic | | | | | | |
| | | Acid | | | | | | |
| | MT-500 | Titanium Dioxide | Presperse | 0.00 | 0.00 | 0.00 | 0.00 | 2.00 |
| | NS PGPR | Polyglyceryl 3 | Next Step | 0.30 | 0.30 | 0.30 | 0.30 | 0.30 |
| | | Polyricinoleate | Lab | | | | | |
| | | | | 100.00 | 100.00 | 100.00 | 100.00 | 100.00 |

### Lab Procedure

1. Combine phase A ingredients and lightning mix at 500 rpm for 5 minutes and heat to 80 C
2. Add phase B to phase A, and lightning mix at 300-500 rpm for 15 minutes to break up agglomerates then homomix for 15 minutes
3. Add phase C ingredients to phases AB with homomixing at 3,000-3,500 rpm. Allow to mix for 10-15 minutes at 80 C
4. Pour into moulds

Set S.

| Formula: | | | | | | | | |
|---|---|---|---|---|---|---|---|---|
| Product: SPF 50 Light lotion | | | | | | | | |
| Note: this a W/O emulsion | | | | 1 | 2 | 3 | 4 | 5 |
| Phase | Tradename | Description | Supplier | % | % | % | % | % |
| A | NS Vsol-5 | Isododecane, | Next | 12.00 | 12.00 | 12.00 | 12.00 | 10.00 |
| | | Hydrogenated | Step Lab | | | | | |
| | | Polydecene, | | | | | | |
| | | Dimethicone | | | | | | |
| | Jeesile IDD | Isododecane, | Jeen | 6.40 | 6.40 | 6.40 | 6.40 | 6.40 |
| | | Dimethicone | | | | | | |
| | | Crosspolymer-3 | | | | | | |
| | 5 CST | Dimethicone | Any | 9.50 | 9.50 | 9.50 | 9.50 | 9.50 |
| | Dermol TDSA | Tridecyl Salicylate | ALZO | 5.30 | 5.30 | 5.30 | 5.30 | 5.30 |
| | SolarYze | Oryza sativa (rice) | Next | 2.00 | 2.00 | 2.00 | 2.00 | 2.00 |
| | | bran extract | Step Lab | | | | | |
| | Caprylyl | Caprylyl Glycol | Any | 0.25 | 0.25 | 0.25 | 0.25 | 0.25 |
| | Glycol | | | | | | | |
| | Phenoxyethanol | Phenoxyethanol | Any | 0.35 | 0.35 | 0.35 | 0.35 | 0.35 |
| B | NS Plant Gel | Dodecane (and) | Next | 10.00 | 10.00 | 10.00 | 10.00 | 10.00 |
| | 2002 | Stearalkonium | Step Lab | | | | | |
| | | Bentonite (and) | | | | | | |
| | | Triethyl Citrate | | | | | | |
| | Abil EM 90 | Cetyl PEG/PPG-10/1 | Evonik | 3.00 | 3.00 | 3.00 | 3.00 | 3.00 |
| | | Dimethicone | | | | | | |
| | NS Gel IDD | Isododecane(and) | Next | 6.40 | 6.40 | 6.40 | 6.40 | 6.40 |
| | | Quaternium-90 | Step Lab | | | | | |
| | | Bentonite (and) | | | | | | |
| | | Propylene Carbonate | | | | | | |
| C | SuperZinc ® | Zinc Oxide, Ethyl | Vizor | 17.00 | 15.00 | 0.00 | 10.00 | 2.00 |
| | 1000-18 | Ferulate, | | | | | | |
| | | Triethoxystearyl | | | | | | |
| | | Silane | | | | | | |
| | Super Ti 1000- | Titanium Dioxide, | Vizor | 0.00 | 2.00 | 17.00 | 10.00 | 17.00 |
| | 18 | Ethyl Ferulate, | | | | | | |
| | | Triethoxystearyl | | | | | | |
| | | Silane | | | | | | |
| | MT-500 | Titanium Dioxide | Presperse | 0.00 | 0.00 | 0.00 | 0.00 | 0.00 |
| | NS PGPR | Polyglyceryl 3 | Next | 0.30 | 0.30 | 0.30 | 0.30 | 0.30 |
| | | Polyricinoleate | Step Lab | | | | | |
| | NS Spherilon- | Nylon-12 | Next | 1.50 | 1.50 | 1.50 | 1.50 | 1.50 |
| | 10 | | Step Lab | | | | | |
| D | Deionized | Water | Any | 25.00 | 25.00 | 25.00 | 25.00 | 25.00 |
| | Water | | | | | | | |
| | Magnesium | Magnesium Sulfate | Any | 1.00 | 1.00 | 1.00 | 1.00 | 1.00 |
| | Sulfate | | | | | | | |
| | | | | 100.00 | 100.00 | 100.00 | 103.00 | 100.00 |

### Lab Procedure

1. Combine phase A ingredients and lightning mix at 500 rpm for 5 minutes.
2. Add phase B to phase A, and lightning mix at 300-500 rpm for 15 minutes to break up agglomerates
3. Add phase C ingredients to phases AB with homomixing at 3,000-3,500 rpm. Allow to mix for 10-15 minutes. Scrape down the sides of the beaker, and the homogenizer
4. Combine phase D ingredient in a separate beaker, and lightning mix until uniformly dispersed.
5. Add phase D to phases A-C slowly with homomixing at 3,500 rpm-4,000 rpm; continue mixing for 15 minutes
6. Drop batch

Set T.

| Formula: | | | | | | | | |
|---|---|---|---|---|---|---|---|---|
| Product: SPF 50 Light lotion | | | | | | | | |
| Note: this a W/O emulsion | | | | 1 | 2 | 3 | 4 | 5 |
| Phase | Tradename | Description | Supplier | % | % | % | % | % |
| A | NS Vsol-5 | Isododecane, | Next | 12.00 | 12.00 | 12.00 | 12.00 | 10.00 |
| | | Hydrogenated | Step Lab | | | | | |
| | | Polydecene, | | | | | | |
| | | Dimethicone | | | | | | |
| | Jeesile IDD | Isododecane, | Jeen | 6.40 | 6.40 | 6.40 | 6.40 | 6.40 |
| | | Dimethicone | | | | | | |
| | | Crosspolymer-3 | | | | | | |
| | 5 CST | Dimethicone | Any | 8.50 | 8.50 | 8.50 | 5.50 | 8.50 |
| | Dermol TDSA | Tridecyl Salicylate | ALZO | 5.30 | 5.30 | 5.30 | 5.30 | 5.30 |
| | SolarYze | Oryza sativa (rice) | Next | 2.00 | 2.00 | 2.00 | 2.00 | 2.00 |
| | | bran extract | Step Lab | | | | | |
| | Caprylyl | Caprylyl Glycol | Any | 0.25 | 0.25 | 0.25 | 0.25 | 0.25 |
| | Glycol | | | | | | | |
| | Phenoxyethanol | Phenoxyethanol | Any | 0.35 | 0.35 | 0.35 | 0.35 | 0.35 |
| B | NS Plant Gel | Dodecane (and) | Next | 10.00 | 10.00 | 10.00 | 10.00 | 10.00 |
| | 2002 | Stearalkonium | Step Lab | | | | | |
| | | Bentonite (and) | | | | | | |
| | | Triethyl Citrate | | | | | | |
| | Abil EM 90 | Cetyl PEG/PPG-10/1 | Evonik | 3.00 | 3.00 | 3.00 | 3.00 | 3.00 |
| | | Dimethicone | | | | | | |
| | NS Gel IDD | Isododecane(and) | Next | 6.40 | 6.40 | 6.40 | 6.40 | 6.40 |
| | | Quaternium-90 | Step Lab | | | | | |
| | | Bentonite (and) | | | | | | |
| | | Propylene Carbonate | | | | | | |
| C | SuperZinc ® | Zinc Oxide, Ethyl | Vizor | 17.00 | 15.00 | 0.00 | 10.00 | 2.00 |
| | 1000-P | Ferulate, | | | | | | |
| | | Phenyltriethoxy | | | | | | |
| | | Silane | | | | | | |
| | Super Ti 1000- | Titanium Dioxide, | Vizor | 0.00 | 2.00 | 17.00 | 10.00 | 17.00 |
| | P2 | Ethyl Ferulate, | | | | | | |
| | | Diphenyldiethoxy | | | | | | |
| | | Silane | | | | | | |
| | MT-500 | Titanium Dioxide | Presperse | 1.00 | 1.00 | 1.00 | 1.00 | 1.00 |
| | NS PGPR | Polyglyceryl 3 | Next | 0.30 | 0.30 | 0.30 | 0.30 | 0.30 |
| | | Polyricinoleate | Step Lab | | | | | |
| | NS Spherilon- | Nylon-12 | Next | 1.50 | 1.50 | 1.50 | 1.50 | 1.50 |
| | 10 | | Step Lab | | | | | |
| D | Deionized | Water | Any | 25.00 | 25.00 | 25.00 | 25.00 | 25.00 |
| | Water | | | | | | | |
| | Magnesium | Magnesium Sulfate | Any | 1.00 | 1.00 | 1.00 | 1.00 | 1.00 |
| | Sulfate | | | | | | | |
| | | | | 100.00 | 100.00 | 100.00 | 100.00 | 100.00 |

### Lab Procedure

1. Combine phase A ingredients and lightning mix at 500 rpm for 5 minutes.
2. Add phase B to phase A, and lightning mix at 300-500 rpm for 15 minutes to break up agglomerates
3. Add phase C ingredients to phases AB with homomixing at 3,000-3,500 rpm. Allow to mix for 10-15 minutes. Scrape down the sides of the beaker, and the homogenizer
4. Combine phase D ingredient in a separate beaker, and lightning mix until uniformly dispersed.
5. Add phase D to phases A-C slowly with homomixing at 3,500 rpm-4,000 rpm; continue mixing for 15 minutes
6. Drop batch

Set U.

| Formula: | | | | | | | | |
|---|---|---|---|---|---|---|---|---|
| Product: SPF 50 | | | | | | | | |
| Stick | | | | 1 | 2 | 3 | 4 | 5 |
| Phase | Tradename | Description | Supplier | % | % | % | % | % |
| A | Dermol IPP | Isopropyl Palmitate | Alzo | 9.00 | 9.00 | 9.00 | 11.90 | 11.00 |
| | Neobee M | Caprylic/Capric | Stepan | 16.90 | 16.90 | 16.90 | 11.00 | 12.90 |
| | | Triglyceride | | | | | | |
| | Dermol 816 | Ethyl Hexyl Palmitate | Alzo | 20.00 | 20.00 | 20.00 | 20.00 | 20.00 |
| | 5 CST | Dimethicone | Any | 9.50 | 9.50 | 9.50 | 9.50 | 9.50 |
| | Dermol | Tridecyl Salicylate | ALZO | 5.30 | 5.30 | 5.30 | 5.30 | 5.30 |
| | TDSA | | | | | | | |
| | SolarYze | Oryza sativa (rice) | Next Step | 2.00 | 2.00 | 2.00 | 2.00 | 2.00 |
| | | bran extract | Lab | | | | | |
| B | SP-63 | Carnauba Wax | Strah&Pitsch | 2.00 | 2.00 | 2.00 | 2.00 | 2.00 |
| | Jeenate 3H | Polyethylene | Jeen | 6.00 | 6.00 | 6.00 | 6.00 | 6.00 |
| | NS Gel | Octyldodecanol, | Next Step | 10.00 | 10.00 | 10.00 | 10.00 | 10.00 |
| | EUG | Quaternium-90 | Lab | | | | | |
| | | Bentonite, Propylene | | | | | | |
| | | Carbonate | | | | | | |
| C | SuperZinc ® | Zinc Oxide, Ethyl | Vizor | 17.00 | 15.00 | 0.00 | 10.00 | 2.00 |
| | 1000-P | Ferulate, | | | | | | |
| | | Phenyltriethoxy | | | | | | |
| | | Silane | | | | | | |
| | Super Ti | Titanium Dioxide, | Vizor | 0.00 | 2.00 | 17.00 | 10.00 | 17.00 |
| | 1000-P2 | Ethyl Ferulate, | | | | | | |
| | | Diphenyldiethoxy | | | | | | |
| | | Silane | | | | | | |
| | Super Ti | Titanium Dioxide, | Vizor | 2.00 | 0.00 | 0.00 | 2.00 | 0.00 |
| | 1000 | Ethyl Ferulate, | | | | | | |
| | | Triethoxycapryl | | | | | | |
| | | Silane | | | | | | |
| | Super Ti | Titanium Dioxide, | Vizor | 0.00 | 2.00 | 2.00 | 0.00 | 0.00 |
| | Natural | Polyhydroxystearic | | | | | | |
| | | Acid | | | | | | |
| | MT-500 | Titanium Dioxide | Presperse | 0.00 | 0.00 | 0.00 | 0.00 | 2.00 |
| | NS PGPR | Polyglyceryl 3 | Next Step | 0.30 | 0.30 | 0.30 | 0.30 | 0.30 |
| | | Polyricinoleate | Lab | | | | | |
| | | | | 100.00 | 100.00 | 100.00 | 100.00 | 100.00 |

### Lab Procedure

1. Combine phase A ingredients and lightning mix at 500 rpm for 5 minutes and heat to 80 C
2. Add phase B to phase A, and lightning mix at 300-500 rpm for 15 minutes to break up agglomerates then homomix for 15 minutes
3. Add phase C ingredients to phases AB with homomixing at 3,000-3,500 rpm. Allow to mix for 10-15 minutes at 80 C
4. Pour into moulds

The formulations should exhibit the properties of good wear, minimal migration, and high shine when applied on lips and which last for at least 2 hours.

While the present compositions and methods have been described with reference to the specific variations thereof, it should be understood by those skilled in the art that various changes may be made, and equivalents may be substituted without departing from the true spirit and scope of the compositions and methods described herein. In addition, many modifications may be made to adapt a particular situation, material, composition of matter, process, process step or steps, to the objective, spirit and scope of the compounds and methods described herein. All patents and publications cited above are hereby incorporated by reference.

Where a specific numerical value is mentioned herein, it will be understood by those of skill in this art that the values, radios, and ranges may be increased or decreased by 20%, without departing from the scope and teachings of the present application, unless some different range is specifically mentioned. Where a specified exemplary formulation is provided with component amounts it will be understood by those of skill in this art that adaptive and combinative arrangements will be further understood and are also intended to be encompassed.

Having described at least one of the preferred embodiments of the present invention, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. An ultraviolet radiation sun protective composition, comprising:
micronized metal oxide inorganic particles selected from the group consisting of zinc oxide, titanium oxide, cerium oxide and mixtures thereof, the inorganic particles being coated with:
(i) a poly[C8-C20 hydroxycarboxylic acid], the coated particles measured at a 10% loading in dodecane and at 1 minute elapsed time having a Zeta Potential ranging from 2 to 10 my, particularly from 2 to 5 my, amounts of the poly[C8-C20 hydroxycarboxylic acid] to the inorganic particles being in a relative weight ratio of 1:100 to 1:10; or
(ii) an adduct of a hydroxy cinnamate and a silanol, amounts of the adduct to the inorganic particles being in a relative weight ratio of 1:200 to 1:4; or
(iii) mixtures of (i) and (ii).

2. A method of manufacturing the ultraviolet radiation sun protective compositions according to claim 1.

3. A method of minimizing a skin-damaging event by application of a sun protective composition containing an efficacious amount of an ultraviolet radiation sunscreen protective composition according to claim 1.
